(19) United States
(12) Patent Application Publication
(10) Pub. No.: US 2010/0047320 A1
(43) Pub. Date: Feb. 25, 2010

(54) ORAL POLYMERIC MEMBRANE FERULOYL ESTERASE PRODUCING BACTERIA FORMULATION

(76) Inventors: Satya Prakash, Brossard (CA); Jasmine Rohinton Bhathena, Ottawa (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/440,550

(22) PCT Filed: Sep. 7, 2007
(86) PCT No.: PCT/CA07/01583

§ 371 Date: Sep. 30, 2009

Related U.S. Application Data
(60) Provisional application No. 60/824,793, filed on Sep. 7, 2006.

(51) Int. Cl. A61K 35/74; A61K 35/66; A61K 9/50; A61P 1/00; A61P 1/14; A61P 1/16; A61P 3/00; A61P 3/08; A61P 3/06; A61P 3/10

(52) U.S. Cl. 424/439; 424/93.1; 424/490; 424/93.2; 424/93.4; 424/93.51; 424/93.45

(57) ABSTRACT

The present invention relates to an oral formulation to lower serum or hepatic lipid and triglyceride concentrations, hepatic inflammation and/or insulin resistance in a patient comprising live feruloyl esterase producing microorganisms alone or in association with a pharmaceutically acceptable carrier resistant to gastric conditions, and wherein the microorganisms are wild type, genetically modified, or combination thereof. The present invention is also directed to an oral formulation to lower serum or hepatic lipid and triglyceride concentrations, hepatic inflammation and/or insulin resistance in a patient, which comprises polymeric microcapsules containing live feruloyl esterase producing microorganisms in suspension in a pharmaceutically acceptable carrier, wherein said microcapsules are semipermeable and resistant to gastro-intestinal conditions, and wherein said microorganism are wild type, genetically modified, or combination thereof as well as methods of preventing or improving liver diseases and disorders and uses thereof.

### FIELD OF THE INVENTION

The present invention relates to an oral formulation to lower serum or hepatic lipid and triglyceride concentrations, hepatic inflammation and/or insulin resistance in a patient, methods of preventing and /or reducing liver diseases and/or disorders and uses thereof.

### BACKGROUND OF THE INVENTION

Non-alcoholic fatty liver disease (NAFLD) is a condition that is becoming increasingly recognized worldwide due to its prevalence in obesity, diabetes, and insulin resistance syndrome. It is a progressive disease and one of the leading causes of liver cirrhosis and an emerging factor in hepatocellular cancer. A recent analysis of the National Health and Nutritional Evaluation Survey (NHANES III) suggests that 10-24% of American adults have NAFLD, making NAFLD three times more common than diabetes mellitus and 5-10 times more common than chronic hepatitis C. Other large, population based surveys in Europe and Japan are in agreement regarding the high prevalence of this disorder NAFLD occurs commonly in diabetics and the obese: 21-78% of diabetics, 57-74% of obese persons, and 90% of morbidly obese persons are affected. NAFLD also occurs in children: 2.6% of normal weight children and up to 52.8% of obese children have been diagnosed with fatty liver disease. NAFLD is thus one of the most widespread chronic diseases in the world, which imposes a substantial expense on the public as well as on patients of NAFLD and their families.

NAFLD refers to a group of conditions where there is accumulation of excess fat in the liver of people who drink little or no alcohol. The most common form of NAFLD is a non-serious condition called fatty liver. In fatty liver, fat accumulates in the liver cells. A small group of people with NAFLD may have a more serious condition named non-alcoholic steatohepatitis (NASH). In NASH, fat accumulation is associated with liver cell inflammation and different degrees of scarring. NASH is a potentially serious condition that may lead to severe liver scarring and cirrhosis. Cirrhosis occurs when the liver sustains substantial damage, and the liver cells are gradually replaced by scar tissue (see figure), which results in the inability of the liver to work properly. Some patients who develop cirrhosis may eventually require a liver transplant.

Less is known about what causes NASH to develop. Researchers are focusing on several factors that may contribute to the development of NASH. These include i) Oxidative stress (imbalance between pro-oxidant and anti-oxidant chemicals that lead to liver cell damage), ii) Production and release of toxic inflammatory proteins (cytokines) by the patient's own inflammatory cells, liver cells, or fat cells and iii) Liver cell necrosis or death, called apoptosis.

An article, entitled “NAFLD, NASH and now NAS”, by S. Komacki and A. B. West, Adv. Anat. Pathol., volume 13, number 2, 2006., describes the various stages of NAFLD and establishes the criteria to follow in order to diagnose a NASH, border-line NASH and not NASH condition in a patient. Briefly, based on the evaluation of 50 liver biopsies, pathologist from 9 different medical centers and mandated by the National Institute of Diabetes & Digestive & Kidney Disease, instituted criteria on categories of potentially reversible injury to be evaluated and graded. Such grade is entitled NAFLD Activity Score or NAS. The criteria are the following: steatosis (score 0 to 3), lobular inflammation (score 0 to 3) and ballooning degeneration (score 0 to 2), with a final score of 0 to 8. A total score of 0 to 2 is considered not diagnostic of NASH, a score of 5 is diagnosed as NASH whereas a score of 3 or 4 is either diagnosed as NASH, borderline NASH and not NASH depending on the situation. This classification differs in various ways from the scoring system of Brunt and his colleagues, established in 1999. Indeed, minimal steatosis was defined as less than 5% whereas mild steatosis represents 5% to 33%. Ballooning degeneration of hepatocytes is limited to three categories: non, few and many. For staging of NASH, stage 1 fibrosis is subdivided into delicate (stage 1A) and dense (stage 1B) perisinusoidal fibrosis as well as portal and periportal fibrosis (stage 1C). In conclusion, the NAS score has to be used in conjunction with the overall clinicopathologic evaluation of a patient's condition to allow the granting of a diagnostic of value. However, even if it could be of interest in diagnosing the disease, such distinctions between a NASH-type disorder, a non-NASH-type disorder and a borderline-NASH disorder have to be put aside when elaborating and selecting a therapy, an effective composition being applicable to all of the above as well as to NAFLD.

Hepatic steatosis (or fatty liver) is defined as the excessive accumulation of lipids in the hepatocytes, with “excessive accumulation” meaning lipid accumulation exceeding the normal 5% of the weight of the liver and commonly causes limited increases in serum aminotransferases (less than 4 times the upper limit of the norm). In macrovesicular hepatic steatosis, large droplets of triglycerides swell the hepatocytes, displacing their nucleus towards the periphery of the cells, as occurs in adipocytes. In microvesicular hepatic steatosis, small droplets of triglycerides accumulate in the hepatocytes, leaving the nuclei in a central position, and the hepatocytes then assume a foamy appearance.

For an exhaustive review of the pathogenesis, clinical aspects, diagnosis and treatment of NASH, see Sheth S. G. et al. (Non-alcoholic steatohepatitis, Ann. Intern. Med. 1997, 127-137). Various treatment strategies such as weight loss and/or exercise, thiazolidinediones, metformin, lipid-lowering agents (statins) and antioxidants have been studied (Tilg, H. & Kaser, A. Treatment strategies in nonalcoholic fatty liver disease. Nature Clinical Practice Gastroenterology & Hepatology 2, 148-155 (2005)). Although beneficial, these methods pose several limitations. For example, the success of diet, exercise, and behavior management relies heavily on the strict compliance of the affected individual. Also, conflicting data on the therapeutic efficacy of the above mentioned drugs have been reported in the literature. In addition, article entitled “Treatment of nonalcoholic fatty liver disease” by J. Siebler and P. R. Galle published in the World Journal of Gastroenterology, 2006, focuses on the evaluation of the various non-alcoholic fatty liver disease's available treatments and their limitations and need for developing other methods.

There are several known risk factors associated with fatty liver diseases. Insulin resistance and obesity represent the most important risk factors for the development of NAFLD and the progression to its aggravated forms, non-alcoholic steatohepatitis (NASH), fibrosis, cirrhosis and hepatocellular carcinoma. The precise role of a drug-free management for improving insulin resistance and NASH is studied in comparison with the review of recent medical treatments. These studies indicate the idea that prescription drugs are not the optimal solution to the disease's treatment and research should focus on prevention and effective management of risk factors such as obesity and insulin resistance. There are various treatment modalities such as insulin sensitizers, weight reduction, enhanced physical activities, jejuno-ileal bypass, pharmacological agents, ursodeoxycholic acid treatments, lipid lowering drugs such as statins, TNF-A blockers, triglyceride lowering drugs has been proposed. However, these methods have various limitations. For example, the first option for a patient with a body mass index (BMI) of less than 25 kg/m² consists of a simple reduction of body weight which is not feasible as an effective treatment methods. Similarly, jejuno-ileal bypass or very low energy diets (<500 kcal daily) are not recommended because of exacerbation of steatohepatitis, a combination of a restricted calorie intake and physical exercise have shown improvement in insulin resistance. However, it did not improved liver histology; a concrete measure of effective therapy methods. In addition, in the case of patients with a BMI of more than 35 kg/m², more aggressive weight reduction and gastric bypass surgery is needed which is a complicated, expensive and risky procedure. In addition, side-effects such as worsening of liver condition in patients taking medication or submitted to surgery have been reported.

Another possible method of treatment consists of the intake of antioxidants such as vitamin E and C along with a reduced diet and exercise. However, the results were not encouraging to be adopted as therapy agent.

Treatment with ursodeoxycholic acid (UDCA) was proposed. However, no successful treatment was observed in clinical trials compared to control group.

Insulin sensitizer has been proposed as a possible therapeutic interventions. For example, metformin showed beneficial effects which were not long lasting resulting in relapse of the disease. Another class sensitizer thiazolinediones were proposed. However, although this compound had interesting properties, the Food and Drug Administration (FDA) did not approved its wide uses because of serious associated hepatotoxicity and other various adverse effects such as weight gain and increase in total body adiposity.

Lipid lowering drugs are also considered a possible treatment for NAFLD. Gemfibrozil was tested in NASH suffering patients but only a reduction in alanine aminotransferase (ALT) was observed. Statins are another potential treatment, however very limited results do not support the use of these in therapy for NAFLD. Use of oligosaccharides, described in U.S. Pat. No. 6,083,927 entitled “Hepatic disturbance improver”, to obviate the problems associated with conventional methods of improving fatty liver by providing a hepatic disturbance improver for reducing fat in hepatocytes. However, its clinical efficacy is yet to be established.

United States Patent Application No. US 2004/0029805 A1 entitled “Prevention and treatment of nonalcoholic fatty liver disease (NAFLD) by antagonism of the receptor to glucose-dependant insulinotropic polypeptide (GIP)” treats of the use of various forms of GIP-receptor antagonists in order to limit the response of insulin to GIP after an intake of food, thereby preventing and treating NAFLD through avoidance of a rise of insulin resistance and of hypeinsulinemia. The GIP is a hormone playing a major role in maintaining the glucose balance following high glucose and fat meals. Such receptor can be antagonized through the use of various peptide receptor antagonists such as GIP (NH2), non-peptide receptor antagonists or through the use of antisense recombinant technology, either accomplished by injection, oral administration or gene therapy. A therapeutic composition can be elaborated for the delivery of the compounds; such pharmaceutical composition can contain one or more antagonists in a pharmaceutically acceptable carrier. Other components, such as flavor, color and preservative can also be added if no interference with the antagonists is created. The GIP receptor antagonist, in doses varying from 0, 1 nM to 100 M in the pharmaceutical composition, can be administered by parental, gene therapy, topical, oral, rectal or nasal route based on the type of carrier. However, even though GIP receptor inhibitors appear as effective compounds in lowering insulin resistance and hyperlipidemia, the proposed methods of administration are not optimal. Indeed, it has been well demonstrated that most of the cited routes lead to degradation of the compounds due to internal degradation such as enzymatic destruction, hard incorporation of the compound in the blood, and malabsorption.

The article entitled “Treatment of non-alcoholic fatty liver disease” by L. A. Adams and P. Angulo, Postgrad. Med. J. 2006;82;315-322 states that primary stages of non-alcoholic fatty liver disease (NAFLD) is mainly associated to insulin resistance accompanying obesity, diabetes and hyperlipidemia. However, several pathogenic factors, called “the second hit”, aggravate the situation and engender hepatic damages. These secondary pathogenic causes, such as glucose intolerance, hypertriglyceridemia and rapid weight loss, are the ones to be controlled and treated. When a diagnosis of NAFLD is confirmed through various biopsies, ultrasound, computed tomography or magnetic resonance, various treatments are available. First, associated metabolic conditions, such as hypertension, central obesity and low HDL cholesterol, have to be evaluated in NAFLD suffering patient because their effective management leads to an amelioration of the vascular risk as well as improvement of the disease. Weight loss associated with exercises constitutes a second possibility in order to improve insulin sensibility, thus ameliorating the NAFLD's state: indeed, liver biochemistry and hepatic steatosis showed improvement. However, very severe diets and rapid weight loss are both precarious for one's health as well as hard to follow. In order to facilitate such treatment, pharmacotherapeutic agents have been developed to assist weight loss: such compounds comprise lipase inhibitors (orlistat), anorectic drugs, and sibutramine. However, those drugs all presented important negative side-effects and many of them had to be withdrawn from the market. On the other hand, bariatric surgery among morbidly obese patients has been successful in lowering alanine aminotransferase (ALT) levels as well as steatosis: yet, cases of hepatic fibrosis and cirrhosis were linked to such procedure. NAFLD's management can also be effectuated through insulin sensitizing drugs. Insulin resistance is well known to be linked to NAFLD and through lipid accumulation in the liver and, if non-treated, progression of the disease to NASH. Metformin is an agent which has been widely used in those therapies: although improvement of ALT and TNFα's levels and steatosis, treatment is still not safe, lactic acidosis being a feared complication of metformin's therapy. A second type of insulin sensitizing drug are thiozoladinediones: recognized as having the power to both lower insulin resistance and liver fibrosis, they were however removed from the market because of idiosyncratic liver toxicity.

Pioglitazone and rosiglitazone also showed improvement in ALT levels, hepatic steatosis and hepatic inflammation. Negative side-effects such as weight gain with fat redistribution and hepatotoxicity were noticed. Antioxidants represent another possible source of treatment, since an impressive level of oxidative stress and lipid peroxydation characterizes NAFLD. Intake of daily vitamin E was studied but no real evidence if its benefits were found. Moreover, serious side-effects such as heart failure were discovered. A second studied antioxidant agent is probucol: even if NAFLD patient's condition improved, this lipid lowering antioxidant was withdrawn from the market because of pro-arrhythmic potential. A variety of existing hepato-protective agents already used in various liver diseases were tested on NAFLD. Pentoxyfilline, losartan, ursodeoxycholic acid (UDCA) and intestinal derived bacterial endotoxin all induced lowering of either steatosis, inflammation and fibrosis: still, the results aren't significant enough because of limited trials and because various liver injury and toxicity. Finally, lipid-lowering drugs were tested, as hypertriglycemia and low HDL cholesterol levels are a manifestation of insulin resistance and frequent among NAFLD patients. Previously removed from the market, statin drugs were shown to be non-toxic in patients with raised liver enzymes. Nevertheless, the limited amount of patients who participated in theses trials along with the non-significant improvement of NAFLD patient's condition proves that the use of such compound is not optimal.

Another treatment method such as blockade of TNF-α, a biologically active molecule produces by adipose tissue was proposed in fatty liver disease. However later it was reported that TNF-α is not useful in fatty liver diseases. Adiponectin, a very similar molecule to TNF-α, seems to have very impressive effects. Indeed, it reduces body fat, improves hepatic and peripheral insulin sensitivity as well as decease fatty acid levels and inflammation. Nevertheless, the lack of patients in the study compromises its credibility. Finally, liver transplantation represents a possible answer for the therapy of liver diseases. Patients with simple steatosis have a benign prognosis whereas patients with the possibility to develop cirrhosis and hepatocellular carcinomas in NASH are important. However, patients who underwent such procedure very often developed recurrent NASH, hyperlipidemia, increased body weight, steatosis and steatohepatitis. In such cases, liver transplant may be required which is complicated procedure. Thus, an alternative formulation is desirable.

Probiotics have been proposed as a treatment option because of their modulating effect on the gut flora that could influence the gut-liver axis. The article entitled “Probiotics for non-alcoholic fatty liver disease and/or steatohepatitis.” by Lirussi F, Mastropasqua E, Orando S and Orlando R published in the Cochrane Database of Systemic Reviews, 2007, states that the authors have been unable to identify any meta-analysis or systematic reviews on probiotics for patients with patients with NAFLD and/or NASH (Lirussi, F., Mastropaqua, E., Orando, S. & Orlando, R. Probiotics for non-alcoholic fatty liver disease and/or steatohepatitis. Cochrane Database of Systematic Reviews (2007)). The objective was to evaluate the beneficial and harmful effects of probiotics for non-alcoholic fatty liver disease and/or steatohepatitis. Probiotics might decrease inflammation and therefore improve NAFLD by the following mechanisms (Solga, S. F. & Diehl, A. M. Non-alcoholic fatty liver disease: lumen-liver interactions and possible role for probiotics. Journal of Hepatology 38, 681-687 (2003)): (1) Competitive inhibition and possible exclusion of pathogenic strains of intestinal bacterial overgrowth, especially strains that have lower total in vitro binding capacity. (2) Alteration of the inflammatory effects of pathogenic intestinal bacterial overgrowth through changes in cytokines signaling. (3) Improved epithelial barrier function by modulating cytoskeletal and tight junctional protein phosphorylation). (4) Direct decrease in proinflammatory cytokines, e.g. TNF-alfa. (5) Stimulation of IgA production. The authors searched The Cochrane Hepato-Biliary Group Controlled Trials Register (July 2006), the Cochrane Central Register of Controlled Trials (CENTRAL) in The Cochrane Library (Issue 2, 2006), MEDLINE (1966 to May 2006), and EMBASE (1980 to May 2006). No language restrictions were applied as a search strategy. Randomized clinical trials evaluating probiotic treatment in any dose, duration, and route of administration versus no intervention, placebo, or other interventions in patients with non-alcoholic fatty liver disease were used as a selection criteria. The diagnosis was made by history of minimal or no alcohol intake, imaging techniques showing hepatic steatosis and/or histological evidence of hepatic damage, and by exclusion of other causes of hepatic steatosis. They authors had planned to extract data in duplicate and analyze results by intention-to-treat. No randomised clinical trials were identified. Preliminary data from two pilot non-randomised studies suggest that probiotics may be well tolerated, may improve conventional liver function tests, and may decrease markers of lipid peroxidation. However, effect of probiotics in treating NAFLD is yet to be determined.

In another attempt to use probiotics a pilot study was carried out. In this study entitled “Beneficial effects of a probiotic VSL#3 on parameters of liver dysfunction in chronic liver diseases” by Loguercio C, Federico A, Tuccillo C, Terracciano F, D'Auria M V, De Simone C, Del Vecchio and Blanco C published in the Journal of Clinical Gastroenterology, 2005, the authors evaluated probiotic therapy in patients with various hepatic diseases (Loguercio, C. et al. Beneficial effects of a probiotic VSL#3 on parameters of liver dysfunction in chronic liver diseases. J. Clin. Gastroenterol. 39, 540-543 (2005)) The probiotic mixture was well tolerated in all groups and aminotransferase, GGT, malonildialdehyde plasma levels significantly decreased and the effect was maintained even after one month of washout. However, liver histology was found unchanged indicating the limitation of the VSL#3 treatment.

In another study free bacterial probiotic were examined for their potential in treatment and prevention of fatty liver using gut flora replacement approach. Studies in rodent models of alcoholic fatty liver disease have demonstrated that intestinal bacteria, bacterial endotoxin and TNF-α modulate alcohol-induced liver damage. The concept that intestinal bacteria induce endogenous signals, which play a pathologic role in hepatic insulin resistance and NAFLD, suggests a role for novel probiotic therapy in this not so uncommon condition. Indeed, various rat models of intestinal bacterial overgrowth have been associated with liver lesions similar to NASH, and bacterial overgrowth has been observed significantly more often in patients with NASH compared with control subjects. These links have been explored by Li Z, Yang S, Lin H, Huang J, Watkins P A, Moser A B, Desimone C, Song X Y, Diehl A M in their article entitled “Probiotics and antibodies to TNF inhibit inflammatory activity and improve nonalcoholic fatty liver disease” published in Hepatology, 2003, and by Nardone G, Rocco A in their article “Probiotics: a potential target for the prevention and treatment of steatohepatitis” published in Journal of Clinical Gastroenterology., 2004 (Nardone, G. & Rocco, A. Probiotics: a potential target for the prevention and treatment of steatohepatitis. J. Clin. Gastroenterol. 38, S121-S122 (2004)). However, it is well established in the literature of limitation of available methods to replace gut flora using this conventional probiotic approach.

In another pilot study; “Gut-liver axis: a new point of attack to treat chronic liver damage?” published in the American Journal of Gastroenterology, 2002, Loguercio C, De Simone T, Federico A, Terracciano F, Tuccillo C, Di Chicco M and Carteni M tested a mixture of free different bacteria strains called LAB (Lactobacillus acidophilus, Bifidus, Rhamnosus, Plantarum, Salivarius, Bulgaricus, Lactis, Casei, Breve) associated to fructo-oligo-saccharides as prebiotic, vitamins (B6, B2, B12, D3, C, folic acid), as well as trace elements (Loguercio, C. et al. Gut-liver axis: a new point of attack to treat chronic liver damage? Am. J. Gastroenterol. 97, 2144-2146 (2002)). Three groups of patients were enrolled in the study: 12 patients with biopsy-proven chronic hepatitis C, 10 patients with alcoholic cirrhosis all of whom continued drinking alcohol in excess and 10 patients with biopsy-proven NASH. The NASH patients (all men) were treated with LAB for two months. After treatment, lipid peroxidation indices—malondialdehyde and 4-hydroxinonenal—decreased by 62% and 45%, respectively. TNF-a levels decreased by 18%. However, liver histology, liver function enzymes such as ALT, AST and GGT were found unchanged.

U.S. Pat. No. 6,942,857 entitled “Microorganisms for preventing and/or treating obesity or diabetes mellitus” describes a formulation comprising of microorganisms that are capable of converting oligosaccharides produced by the digestive enzymes into non-digestible polysaccharides, and thereby remarkably reducing the amount of oligosaccharide absorbed into the intestines. This is achieved by providing a pharmaceutical composition comprising at least one of said microorganisms in an amount effective to prevent or treat obesity and diabetes mellitus and a pharmaceutically acceptable carrier. The invention, however, is very specific in dealing with the terms “obesity and “diabetes mellitus” and it does not teaches method to treat fatty liver. Specifically this invention does not show the reduction of elevated hepatic lipid or triglycerides or serum triglycerides or liver enzymes or set the use of the invention in the treatment of NAFLD.

United States Patent Application No. 20070134220 entitled “Lactobacillus fermentum strain and uses thereof” describes an orally delivered ‘medicine’ or pharmaceutical composition comprising a Lactobacillus fermentum strain (LB-f strain) and a pharmaceutically acceptable carrier can potentially be used as a method for treating or preventing various gastrointestinal disorders including ulcers and infections due to Helicobacter pylori, intestinal inflammatory diseases, such as ulcerous colitis, Crohn's disease and pouchitis, irritable bowel syndrome, steatohepatitis, hepatic steatosis, and infectious diarrhoea in mammals, especially humans (Servin, A., Chauviere, G., Polter, M.-H., Le Moal, V. & Gastebois, B. Lactobacillus fermentum strain and uses thereof. 20070134220. 2007). The authors describe that the LB-f strain is required to have capability of preventing colonization of the stomach and the intestine by pathogenic bacteria that are responsible for gastrointestinal disorders and allow re-establishment of the normal gut flora. This patent does not list any in vivo studies to support its claim for the use of this medicine in the treatment of steatosis or NAFLD and limits its conclusions based on in vitro adhesion studies, pathogen diminishing studies and anti-oxidative study data. Therefore a conclusion of this approach efficacy cannot be drawn. Nonetheless, this approach, once again, relies on the traditional dogma of probiotics in which ingesting a smack amount of ‘good’ bacteria supposedly positively alters the colonic microflora for health benefits.

United States Patent Application No. 20060233774 entitled “Composition for the improvement of liver function, the reduction of serum ethanol level and antioxidant activity enhancement” is directed to a composition for use in alcoholic liver function improvement, blood alcohol level reduction and in vivo antioxidant activity enhancement, comprising of Lactobacillus strains and plant extracts along with vitamins. However, neither any specific mechanism nor any liver histology studies indicating effect of the formulation in fatty liver was carried out.

Another United States Patent Application No. 20040248278 entitled “Strain of lactic acid bacterium and edible compositions, drugs and veterinary products containing it” states that an orally or enterally delivered formulation with Streptococcus thermophilus ssp. salivarius strain as an active principle (or as one of the active principles) is effective in the prevention/treatment of hepatic steatosis (fatty liver) and in nonalcoholic hepatic steatosis (De Simone, C. Strain of lactic acid bacterium and edible compositions, drugs and veterinary products containing it. 20040248278. 2004). With this formulation no significant changes were detected in triglycerides, cholesterol and body weight. The subjects were given freeze-dried bacteria in the form of granules. In this patent, authors have relied on the elevation of liver function test enzymes as an indication of NAFLD and their subsequent lowering after treatment as an indication of the efficacy of the treatment. However, the term “liver function” in general refers to a broader normal function of the liver, including, but not limited to, a synthetic function, including, but not limited to, synthesis of proteins such as serum proteins (e.g., albumin, clotting factors, alkaline phosphatase, aminotransferases (e.g., ALT, AST, GGT etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like. Whether a certain method is effective in reducing NAFLD should be determined by any of a number of well-established techniques for measuring liver fibrosis and liver function. Whether NAFLD is reduced is also determined by analyzing a liver biopsy sample and scoring (Brunt (2000) Hepatol. 31:241-246; and METAVIR (1994) Hepatology 20:15-20. Secondary indices of liver function include, but are not limited to, serum transaminase levels, prothrombin time, bilirubin, platelet count, portal pressure, albumin level, and assessment of the Child-Pugh score. Lack of these studies limited the interpretation of the potential of this patent.

In another study inactivated bacterial cells were proposed. United States Patent Application No. 20050180962 entitled “Inactivated probiotic bacteria and methods of use thereof” teaches the use of inactivated probiotic bacteria; and a pharmaceutically acceptable excipient, wherein the bacteria are inactivated by a process other than heating i.e. either gamma irradiation, ultraviolet irradiation or pasteurization; further embodiments of the formulation comprise of an immunosuppressive agent, antibiotic and nutritional beverage comprising nutrients that are readily absorbed by gut epithelium. The inactivated probiotic bacteria of the invention are claimed typically to not elicit an immune response to an antigen of the probiotic bacteria. However, no relevant information of efficacy of these formulations in NAFLD has been established.

U.S. Pat. No. 7,001,756 entitled “Microorganism strain of GM-020 of Lactobacillus rhamnosus and its use for treating obesity” provides a method for treating obesity and complications thereof in a subject comprising administrating said subject with a composition comprising the microorganism strain Lactobacillus rhamnosus GM-020 and an Auricularia polytricha strain. However, this patent does not teach use of polymeric membrane microencapsulated bacteria and any indications of relief from NAFLD.

In other approach lyophilized bacterial mixtures were proposed. U.S. Pat. No. 6,641,808 entitled “Composition for treatment of obesity” describes a probiotic composition comprising a lyophilized culture having Lactobacillus bulgaricus and Streptococcus thermophilus in the treatment of obesity. However, use of Streptococcus bacteria is potentially dangerous. As well, this study does not provide any data as to the clinical efficacy of this formulation in treating NAFLD.

In an attempt to address the lack of suitable formulations use of alternative eukaryotic microorganisms have also been investigated. U.S. Pat. No. 6,753,008 entitled “Dietary supplements beneficial for the liver” describes a composition comprising a plurality of yeast cells, wherein the said plurality of yeast cells are characterized by their ability to normalize the level of serum ALT, alkaline phosphatase (AP), or lactate dehydrogenase 5 (LDH-5) in a mammal with liver problems, said ability resulting from their having been cultured in the presence of an alternating electric field (Cheung, L. Y. Dietary supplements beneficial for the liver. (U.S. Pat. No. 6,753,008). 2004). However, clinical efficacy of this approach yet to be established.

The potential use of live bacterial cells as an approach to prevent or treat NAFLD may be hampered by inherent limitations. For example, of those free bacteria ingested only 1% survive gastric transit limiting the overall therapeutic effect (De Smet et al. 1994). Also, oral administration of live bacterial cells can cause a host immune response, and can be detrimentally retained in the intestine replacing the natural intestinal flora (Taranto et al., 2000; Chin et al., 2000; De Boever and Verstraete, 1999) inducing safety concerns. Furthermore, there are some practical concerns regarding the production, cost, and storage of products containing free bacteria (De Boever and Verstraete, 1999). Thus, concerns of safety and practicality have prevented the regular use of this promising therapy in clinical practice.

Microencapsulation and immobilization patents include U.S. Pat. No. 6,565,777, U.S. Pat. No. 6,346,262, U.S. Pat. No. 6,258,870, U.S. Pat. No. 6,264,941, U.S. Pat. No. 6,217,859, U.S. Pat. No. 5,766,907 and U.S. Pat. No. 5,175,093. It is a technique used to encapsulate biologically active and other materials in specialized ultra thin semi-permeable polymeric membranes. The polymeric membrane protects encapsulated materials from harsh external environments; while at the same time allows the metabolism of selected solutes capable of passing in and out of the microcapsule. In this manner, the live bacteria or yeast can be retained inside and be separated from the external environment and allow targeted deliveries at specific sites. Various studies as well as United States Patent Application No. 20070116671 entitled “Cell and enzyme compositions for modulating bile acids, cholesterol and triglycerides” show that artificial cell Alginate-poly L-lysine-Alginate (APA) microcapsules can be used for oral administration of live bacterial cells (Prakash, S. & Jones, M. L. Cell and enzyme compositions for modulating bile acids, cholesterol and triglycerides. 20070116671. 2007). Therefore, microcencapsulation has been used in this invention.

In summary, NAFLD and NASH are very common liver diseases; several attempts have been made to find suitable therapy methods. Although there have been several promising methods proposed earlier, they are inefficient and associated with several limitations. Therefore, it would be highly desirable to be provided with an oral formulation to lower serum, hepatic lipid and triglyceride concentrations, hepatic inflammation and insulin resistance in a patient which would be safe for oral administration as well as resistant to gastrointestinal conditions.

### SUMMARY OF THE INVENTION

In accordance with the present invention, there is provided a oral formulation containing live feruloyl esterase producing microorganisms (bacteria or yeast), wild type or genetically modified, alone or in combination, free or microencapsulated, capable of reducing serum, hepatic lipid and triglyceride concentrations by metabolizing the diet in the GI tract into free ferulate and free sterols. The free ferulate is then absorbed and could act as an antioxidant within the plasma, and the free sterol inhibits the cholesterol absorption within the GI tract thereby exhibiting clinical benefits. An added advantage of this invention is the increased fecal excretion of cholesterol and its metabolites and ability of the formulation to inhibit liver enzymes due to elevated levels of plasma ferulate. The main objective of the present invention is to provide a pharmaceutical composition comprising at least one of said microorganisms in a pharmaceutically acceptable carrier in an amount effective to prevent or treat NAFLD and NASH. Another objective of the present invention is to provide a food composition containing the microorganisms as an active FAE ingredient.

In accordance with the present invention, there is provided an oral formulation to lower hepatic lipid and triglyceride concentrations, hepatic inflammation and/or insulin resistance in a patient, which comprises free cells or polymeric microcapsules containing live feruloyl esterase producing cells in suspension in a pharmaceutically acceptable carrier, wherein said microcapsules are semipermeable and resistant to gastro-intestinal conditions.

The preferred live feruloyl esterase producing microbial cells are naturally feruloyl esterases producing Lactobacillus, or Bifidobacteria or Bacillus bacterial cells or feruloyl esterase producing genetically engineered cells. Further, Saccharomyces, Schizosaccharomyces, Sporobolomyces, Torulopsis, Trichosporon, Wickerhamia, Candida, Hansenula, Pichia, or Rhodotorula yeast cells and which are natural or genetically modified, principally or in combination.

The “wild type” or naturally feruloyl esterase producing Lactobacillus or Bifidobacteria or Bacillus bacterial cells are chosen from Lactobacillus fermenum 11976, Lactobacillus leichmanni NCIMB 7854, Lactobacillus farciminis NCIMB 11717, Lactobacillus fermentum NCFB 1751, Lactobacillus fermentum NCIMB 2797, Lactobacillus reuteri NCIMB 11951, Bacillus subtilis FMCC 193, Bacillus subtilis FMCC 267, Bacillus subtilis FMCC PL-1, Bacillus subtilis FMCC 511, Bacillus subtilis NCIMB 11034, Bacillus subtilis NCIMB 3610, Bacillus pumilis ATCC 7661, Bacillus sphaericus ATCC 14577 and Bacillus licheniformis ATCC 14580, Bifidobacterium lactis, Bifidobacterium longum, Bifidobacterium bifidum, Bifidobacterium infantis, Bifidobacterium adolescentis, Bifidobacterium angulatum, Bifidobacterium breve, Bifidobacterium catenulatum, Bifidobacterium denticolens, Bifidobacterium dentium, Bifidobacterium gallicum, Bifidobacterium inopinatum, Bifidobacterium pseudocatenulatum, Bifidobacterium lactis, Bifidobacterium minimum, Bifidobacterium subtile, Bifidobacterium thermacidophilum, Bifidobacterium animalis, Bifidobacterium asteroids, Bifidobacterium boum, Bifidobacterium choerinum, Bifidobacterium coryneforme, Bifidobacterium cuniculi, Bifidobacterium gallinarum, Bifidobacterium indicum, Bifidobacterium magnum, Bifidobacterium merycicum, Bifidobacterium pseudolongum subsp. Pseudolongum, Bifidobacterium pseudolongum subsp. Globosum, Bifidobacterium pullorum, Bifidobacterium ruminantium, Bifidobacterium saeculare, Bifidobacterium suis, Bifidobacterium thermophilum.

Other preferred naturally feruloyl esterase producing Lactobacillus or Bacillus or Bifidobacteria bacterial cells are Lactobacillus fermentum 11976 bacterial cells, Lactobacillus fermentum 14932, Lactobacillus reuteri 23272, and Lactobacillus farciminis 29645.

The yeast cells are chosen from Saccharomyces cerevisiae, Saccharomyces carlsbergensis, Saccharomyces chevalieri, Saccharomyces delbrueckii, Saccharomyces exiguous, Saccharomyces fermentati, Saccharomyces logos, Saccharomyces mellis, Saccharomyces oviformis, Saccharomyces rosei, Saccharomyces rouxii, Saccharomyces sake, Saccharomyces uvarum, Saccharomyces willianus, Saccharomyces sp., Schizosaccharomyces octosporus, Schizosaccharomyces pombe, Sporobolomyces roseus, Torulopsis candida, Torulopsis famta, Torulopsis globosa, Torulopsis inconspicua, Trichosporon behrendii, Trichosporon capitatum, Trichosporon cutaneum, Wickerhamia fluoresens, Candida arborea, Candida krusei, Candida lambica, Candida lipolytica, Candida parapsilosis, Candida pulcherrima, Candida rugousa, Candida tropicalis, Candida utilis, Crebrothecium ashbyii, Geotrichum candidum, Hansenula anomala, Hansenula arabitolgens, Hansenula jadinii, Hansenula satumus, Hansenula schneggii, Hansenula subpelliculosa, Kloeckera apiculata, Lipomyces starkeyi, Pichia farinosa, Pichia membranaefaciens, Rhodosporidium toruloides, Rhodotorula glutinis, Rhodotorula minuta, Rhodotorula rubar, Rhodotorula aurantiaca, Saccharomycodes ludwigii, and Saccharomycodes sinenses. For instance, the yeast cells can be of the strain Saccharomyces cerevisiae Hansen AS2.375, AS2.501, AS2.502, AS2.503, AS2.504, AS2.535, AS2.558, AS2.560, AS2.561, AS2.562, or IFFI1048; or Saccharomyces carisbergensis Hansen AS2.420, or AS2.444.

The microcapsules may be made of a material chosen from Alginate-Poly-L-lysine-Alginate [APA], Alginate-Chitosan [AC], Alginate-Chitosan-Polyethylene glycol (PEG)-Poly-L-lysine (PLL)-Alginate [ACPPA], Alginate-Poly-L-lysine-PEG-Alginate [APPA], Alginate-Chitosan-PEG [ACP], Alginate-Poly-L-lysine -Pectinate-Poly-L-lysine-Alginate [APPPA], Genipin cross-linked alginate-chitosan (GCAC).

The microcapsules is preferably made of Alginate-Poly-L-lysine-Alginate [APA] in a preferred embodiment.

The pharmaceutically acceptable carrier may be in the form of a tablet, a capsule, a jellified tablet, a caplet and a liquid formulation. The invention further relates to compositions that contain the feruloyl esterase producing bacteria or yeast cells as active principle, which can assume the form and perform the activity of edible products or dietary supplements, or of a medicine proper, or veterinary products, as a function of the supportive or preventive action, or therapeutic action proper, that the compositions are intended to perform, depending on the particular subjects for whom it is intended.

In accordance with another embodiment of the present invention, there is provided a method for the therapy of a patient suffering from liver diseases and disorders associated with high hepatic lipid and triglyceride concentrations, hepatic inflammation and insulin resistance, which comprises orally administering a sufficient amount of an oral formulation of the present invention. The administration amount can vary depending on the weight and the severity of obesity of the patient, supplemental active ingredients included and microorganisms used therein. In addition, it is possible to divide up the daily administration amount and to administer continuously, if needed. Therefore, range of the administration amount does not limit the scope of the present invention in any way.

In accordance with another embodiment of the present invention, there is provided another composition useful in liver function improvement, and antioxidant activity enhancement in the human body,

It is a further objective of the present invention to provide a health food useful in liver function improvement, antioxidant activity enhancement in the human body, containing the composition as an effective component along with dietary fibre rich in polyphenols and hesperetin metabolites such as ferulic acid (including but not limited to oat bran, wheat bran, whole wheat).

It is a further objective of the present invention to provide a health food useful in liver function improvement, antioxidant activity enhancement in the human body, containing the composition as an effective component along with phytochemicals such as other hydroxycinnamic acids, caffeic acid, sinapic acid present in wine, and chlorogenic acid present in apple.

It is another objective of the present invention to provide a fermented dairy food useful in liver function improvement, containing the composition as an effective component along with probiotics such as inulin, fructooligosaccharide, polydextrose and isomaltooligosaccharides.

It is still a further object of the present invention to provide a health food useful in liver function improvement, blood alcohol level reduction and antioxidant activity enhancement in the human body, containing the composition as an effective component along with psyllium and/or other phytosterols.

The diseases and disorders include non-alcoholic fatty liver disease (NAFLD), alcoholic fatty liver disease (AFLD), liver cirrhosis, liver fibrosis, hyperlipidemia, obesity, type II diabetes, hepatocellular cancer and non-alcoholic steatohepatitis (NASH). With regard to the use of the formulation according to the invention in humans, the preventive or curative action is displayed principally against certain diseases of the liver, such as hepatic steatosis (fatty liver), in particular nonalcoholic hepatic steatosis, and hepatic encephalopathy, against some endocrine and metabolic diseases such as hyperinsulinemia, insulin resistance and obesity. In the case of animals, the veterinary products find useful applications in the treatment of hepatic pathologies and of endocrine and metabolic diseases.

In accordance with another embodiment of the present invention, there is provided the use of a formulation of the present invention for lowering elevated lipid and triglyceride concentrations, hyperlipidemia, hypercholesterolemia, hyperlipoproteinemia and hepatic inflammation and/or insulin resistance in a patient.

In accordance with another embodiment of the present invention, there is provided the use of a formulation of the present invention as an anti-oxidant agent especially in the amelioration of oxidative stress associated diseases such as atherosclerosis, aging etc.

In accordance with another embodiment of the present invention, there is provided the use of a formulation of the present invention as an anti-carcinogenic agent.

In accordance with another embodiment of the present invention, there is provided a method for the therapy of a patient suffering from cancers of the digestive tract (i.e. tongue, esophageal, stomach, intestinal), colorectal cancers, prostate cancer; lung cancer; liver cancer; and breast cancer, which comprises orally administering a sufficient amount of the oral formulation of the present invention.

In accordance with another embodiment of the present invention, there is provided the use of a formulation of the present invention as an anti-tumoral agent.

In accordance with another embodiment of the present invention, there is provided the use of a formulation of the present invention lowering blood pressure.

In accordance with another embodiment of the present invention, there is provided the use of a formulation of the present invention as a neuroprotective agent.

In accordance with another embodiment of the present invention, there is provided a method for the therapy of a patient suffering from diseases chosen from Alzheimer's, cognitive decline, and macular degeneration, which comprises orally administering a sufficient amount of the oral formulation of the present invention.

In accordance with another embodiment of the present invention, there is provided the use of a formulation of the present invention as an agent for prevention of bone degeneration in osteoporosis.

In accordance with another embodiment of the present invention, there is provided the use of a formulation of the present invention as an agent for prevention of menopausal hot flashes.

In accordance with another embodiment of the present invention, there is provided the use of a formulation of the present invention as an agent for prevention of diseases or for enhancing cellular immunity.

In accordance with another embodiment of the present invention, there is provided the use of a formulation of the present invention as an agent to enhance athletic performance.

In accordance with another embodiment of the present invention, there is provided the use of a formulation of the present invention as an agent for renal protective effect and preventive effect on kidney stones.

In accordance with another embodiment of the present invention, there is provided a method for the prevention of renal failure in a patient, which comprises orally administering a sufficient amount of the oral formulation of the present invention.

In accordance with another embodiment of the present invention, there is provided the use of a formulation of the present invention as an agent for treatment of ischemic stroke.

In accordance with another embodiment of the present invention, there is provided the use of a formulation of the present invention to improve brain microcirculation through inhibiting thrombus formation and platelet aggregation as well as blood viscosity.

For the purpose of the present invention, the following terms are defined below.

The term “non-alcoholic fatty liver disease” (NAFLD) is a general pathogenesis of steatosis and cellular injury that isn't alcohol-related. It is a general disease category including various high hepatic lipid concentrations and inflammation related disorders, ranging from simple steatosis, steatosis with nonspecific inflammation to the aggravated condition that is non-alcoholic steatohepatitis (NASH). If non-treated, it can evolve in cirrhosis, fibrosis and hepatocellular cancer.

The term “alcoholic fatty liver disease” (AFLD) is referring to a high hepatic lipid concentrations and inflammation disease consisting of an early and reversible consequence of excessive alcohol consumption.

The term “non-alcoholic steatohepatitis” (NASH) is referring to a common, often “silent” liver disease. It is similar to alcoholic liver disease, but occurs in people who drink little or no alcohol. The major characteristics of NASH are fat in the liver, inflammation and damage. Such condition can lead to cirrhosis.

All references referred herein are hereby incorporated by reference.

### BRIEF DESCRIPTION OF THE DRAWINGS

FIG. 1 illustrates feruloyl esterase activity as detected by the plate-assay method. Various bacteria cells were tested: L. farciminis is on plate A, L. reuteri is on plate B, L. fermentum 11976 is on plate C, L. fermentum 14932 on MRS-EFA (supplemented with 10% w/v in dimethylformamide) is on plate D and MRS-EFA agar as control is on plate E.

FIG. 2 consists of photomicrographs (10×) representing microencapsulated L. fermentum 11976 after exposure to refrigerated storage in (A), to 45 minutes in simulated gastric fluid in (B) and to 45 minutes in simulated gastric fluid followed by 10 hours in simulated intestinal fluid in (C).

FIG. 3 illustrates ferulic acid release due to Lactobacilli FAE activities related to de-esterification of 1.33 mM ethylferulate after 10 hrs. HPLC peak areas of FA indicate FAE activity of L. farciminis microcapsules in (A), of L. reuteri microcapsules in (B), of L. fermentum 11976 microcapsules in (C) and of L. fermentum 14932 microcapsules in (D). Sham microcapsules are used as control.

FIG. 4 consists of photomicrographs of a hamster liver at 6 weeks, the control animal being displayed on top and the test animal on bottom. Picture (A) consists of the photomicrograph of the liver of a control animal on a regular diet: the whole liver is shown on the left whereas a single magnified lobe is shown on the right. Picture (B) consists of the photomicrograph of the liver of the test animal submitted to a lipid diet: the whole liver is shown on the left whereas a single magnified lobe showing lipid deposits in vasculature is shown on the right.

FIG. 5 consists of photomicrographs of a hamster liver at 4 weeks, the control animal being in (A) and the test animal being in (B). In (A), the control animal id fed sham microcapsules and a high lipid diet: single magnified lobe shows excessive lipid deposits in vasculatures. In (B), the test animal is fed microencapsulated L. fermentum 11976 and a high lipid diet: single magnified lobe shows reduced lipid deposits in vasculatures.

FIG. 6A is a graph of a hamster serum total cholesterol profile foe microcapsule oral L. fermentum 11976 formulation treated experimental groups versus sham microcapsule control group after 8 weeks of treatment. The legend is the following: “MC” is for microcapsule and “HL” is for high lipid. FIG. 6B is a graph of hamster serum LDL-Cholesterol profile over 8 weeks for microcapsule oral L. fermentum 11976 formulation treated experimental groups versus sham microcapsule control group. The legend is the following: “MC” is for microcapsule and “HL” is for high lipid.

FIG. 7 is a graph of a hamster serum triglyceride profile over 8 weeks for microcapsule oral L. fermentum 11976 formulation treated experimental groups versus sham microcapsule control group. The legend is the following: “MC” is for microcapsule and “HL” is for high lipid.

FIG. 8 is hamster atherogenic index over 8 weeks for microcapsule oral L. fermentum 11976 formulation treated experimental groups versus sham microcapsule control group. The legend is the following: “MC” is for microcapsule and “HL” is for high lipid.

FIG. 9 is hamster serum blood glucose levels for microcapsule oral L. fermentum 11976 formulation treated experimental groups versus sham microcapsule control group after eight weeks of treatment. The legend is the following: “MC” is for microcapsule and “HL” is for high lipid.

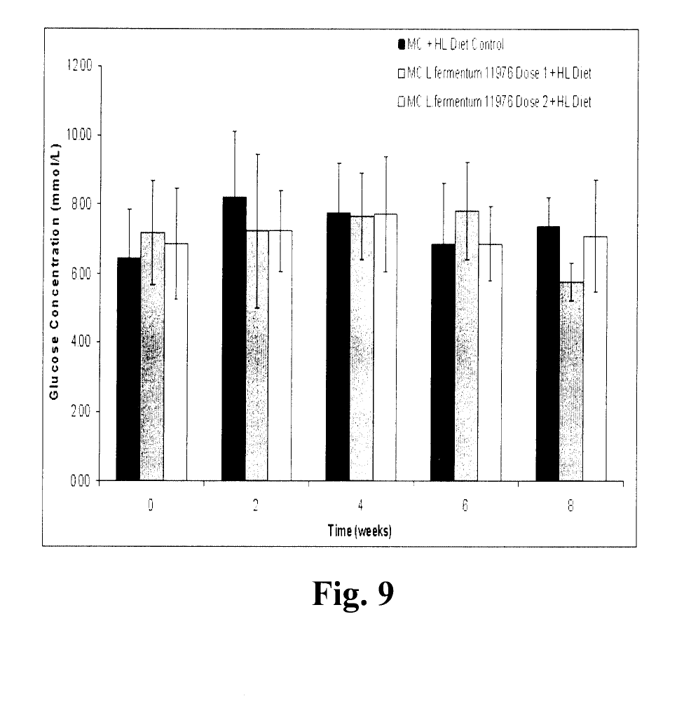

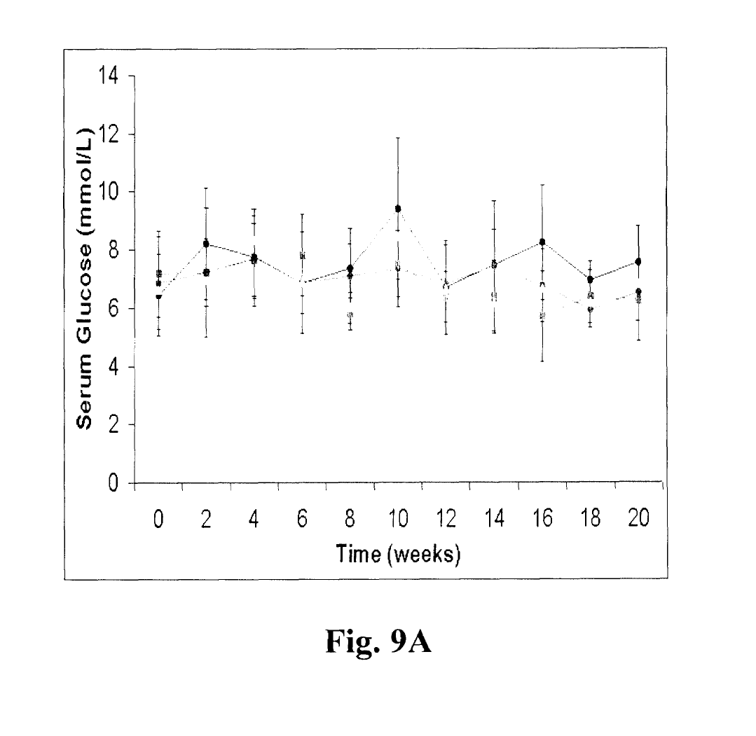

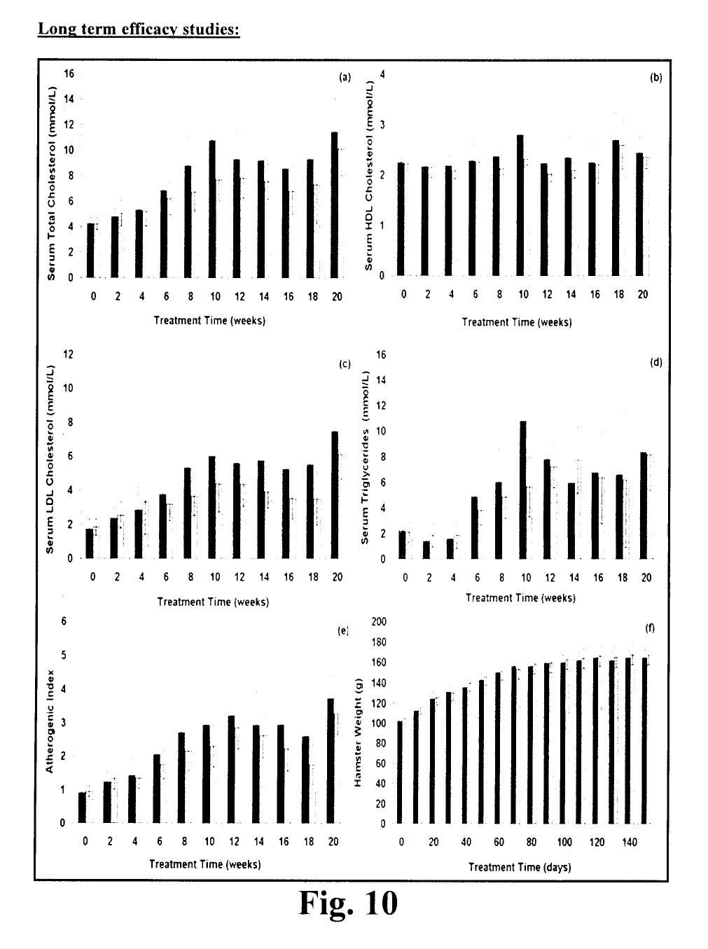

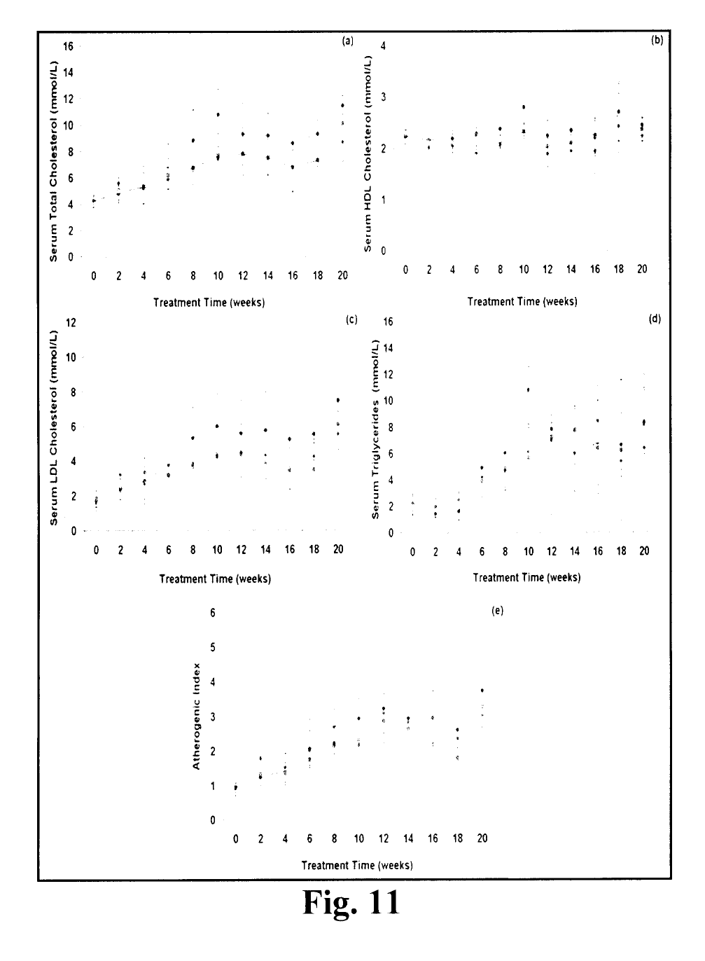

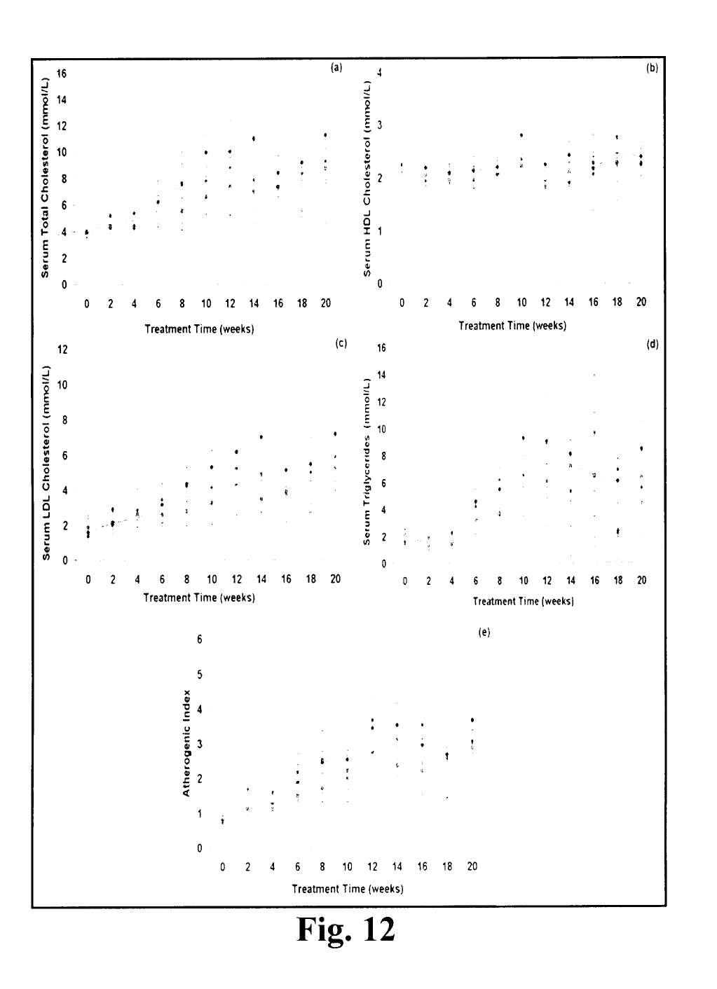

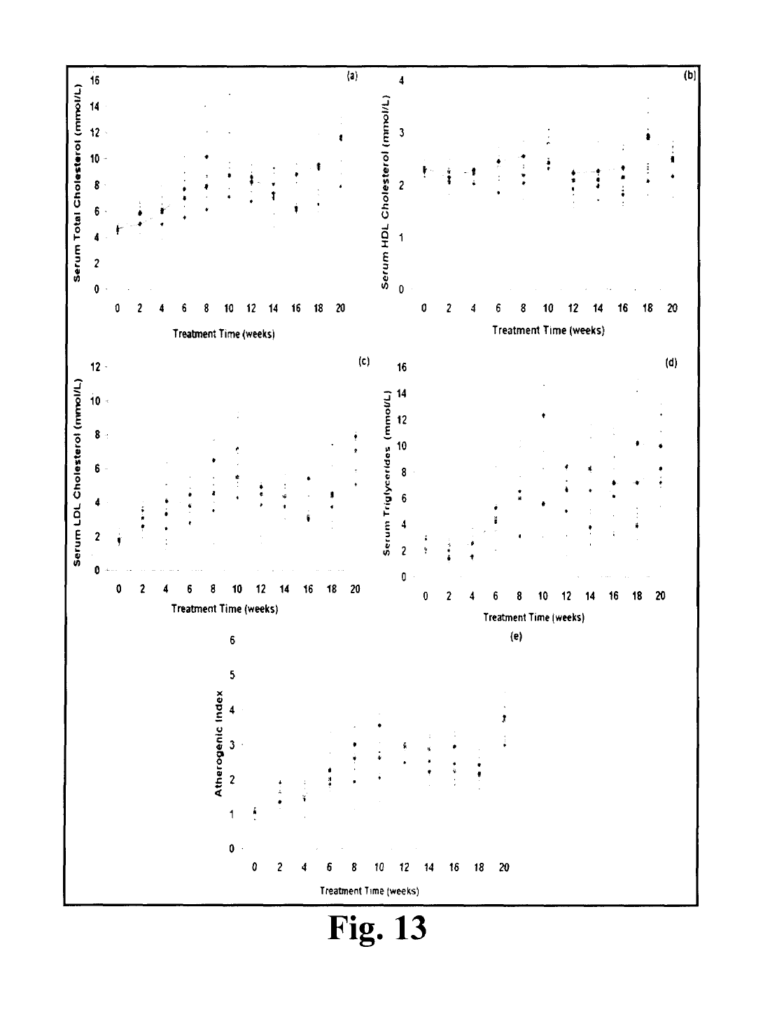

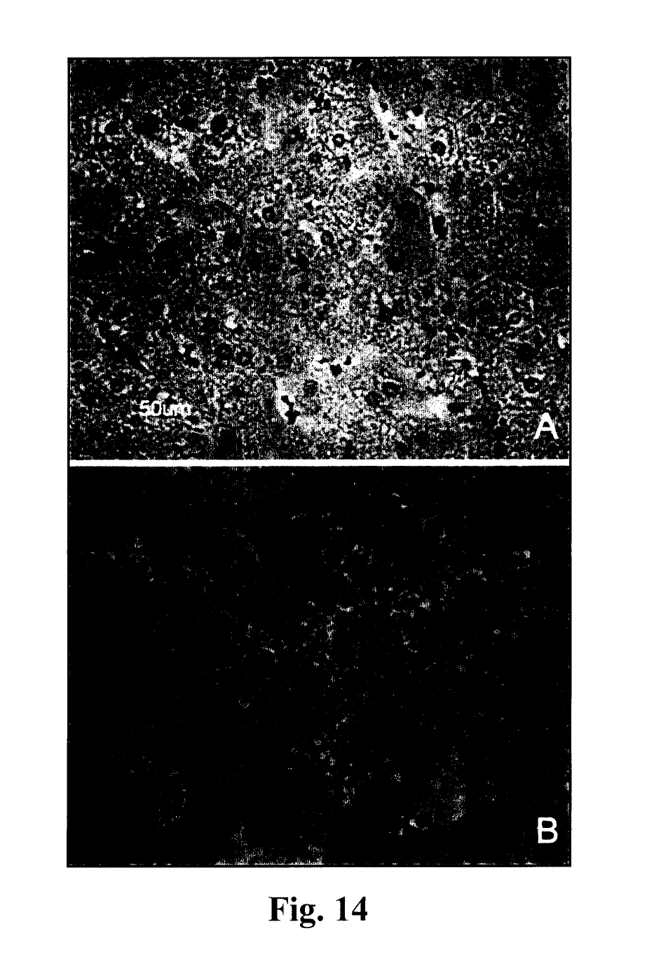

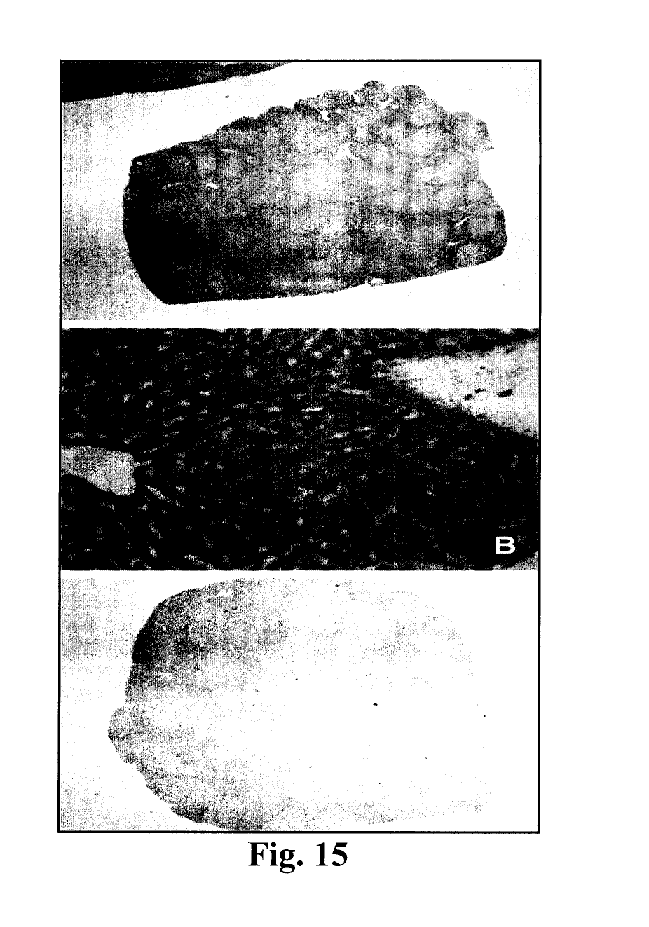

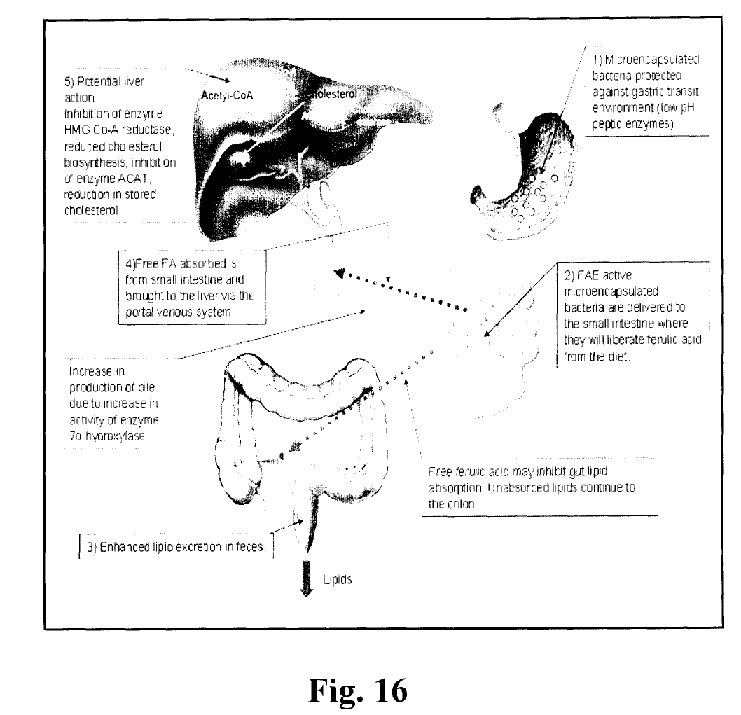

FIG. 9A. Dose dependency of microencapsulated LF11976 formulation for the lowering of serum glucose.
Control-Empty Microcapsules;
[symbol] Treatment D1-Microencapsulated LF11976˜12.51 log cfu/mL
Δ Treatment D2-Microencapsulated LF11976˜12.81 log cfu/mL
Treatment D3-Microencapsulated LF11976˜12.98 log cfu/mL.

FIG. 10. Oral delivery of microencapsulated LF11976 formulation treatment resulted in observable clinical benefits on serum total cholesterol (a), HDL cholesterol (b), LDL cholesterol (c), Triglycerides (d), Al (e). Hamster body weights showed no significant differences between controls and treatment groups (f).
▪ Control-Empty microcapsules
□ Treatment- Microencapsulated LF11976

FIG. 11. Dose dependency of microencapsulated LF11976 formulation for the lowering of serum lipoproteins (a, b, c), lipids (d), and Al (e).
Control-Empty Microcapsules;
[symbol] Treatment D1-Microencapsulated LF11976˜12.51 log cfu/mL
Δ Treatment D2-Microencapsulated LF11976˜12.81 log cfu/mL
D3-Microencapsulated LF11976˜12.98 log cfu/mL.

FIG. 12. Effect of varying dosages of microencapsulated LF11976 treatment on low basal TC (<4.2 mM) hypercholesterolemic hamster serum lipoproteins (a, b, c), lipids (d), and Al (e).
Control-Empty Microcapsules;
[symbol] Treatment D1-Microencapsulated LF11976˜12.51 log cfu/mL
Δ Treatment D2-Microencapsulated LF11976˜12.81 log cfu/mL
Treatment D3-Microencapsulated LF11976˜12.98 log cfu/mL.

FIG. 13. Effect of varying dosages of microencapsulated LF11976 treatment on a population with high basal TC (>4.2 mM): Serum lipoproteins (a, b, c), lipids (d), and Al (e).
Control-Empty Microcapsules;
[symbol] Treatment D1-Microencapsulated LF11976˜12.51 log cfu/mL
Δ Treatment D2-Microencapsulated LF11976˜12.81 log cfu/mL
Treatment D3-Microencapsulated LF11976˜12.98 log cfu/mL.

FIG. 14. Photomicrographs of liver from control hamsters after 20 weeks on a hypercholesterolemic, hyperlipidemic diet (hematoxylin-eosin, (A) 400×, (B) 600×). Hepatocytes are filled with microvasicular fat deposits, leaving the nuclei in a central position, and the hepatocytes have assumed a foamy appearance.

FIG. 15 Photomicrographs of livers from hamsters after 20 weeks on a hypercholesterolemic, hyperlipidemic diet (A) without treatment (gross sample) (Oil Red O, 7.5×), (B) Close up (Oil Red O, 110×) of liver sample from control animal; hepatocytes show microvesicular fat deposition; they are filled with reddish-orange fat deposits, and (C) with FAE producing LF11976 microcapsule formulation oral treatment (Oil Red O, 7.5×).

FIG. 16. Proposed mechanism of action of orally delivered FAE producing lactobacillus formulations in lowering serum lipids for application in treatment of cardiovascular diseases.

### DETAILED DESCRIPTION OF THE INVENTION

In accordance with the present invention, there is provided live feruloyl esterase producing bacteria cells which are naturally feruloyl esterase producing bacterial cells chosen from Lactobacillus leichmanni NCIMB 7854, Lactobacillus farciminis NCIMB 11717, Lactobacillus fermentum NCFB 1751, Lactobacillus fermentum NCIMB 2797, Lactobacillus reuteri NCIMB 11951, Bacillus subtilis FMCC 193, Bacillus subtilis FMCC 267, Bacillus subtilis FMCC PL-1, Bacillus subtilis FMCC 511, Bacillus subtilis NCIMB 11034, Bacillus subtilis NCIMB 3610, Bacillus pumilis ATCC 7661, Bacillus sphaericus ATCC 14577 and Bacillus licheniformis ATCC 14580 Bifidobacterium lactis, Bifidobacterium longum, Bifidobacterium bifidum, Bifidobacterium infantis, Bifidobacterium adolescentis, Bifidobacterium angulatum, Bifidobacterium bifidum, Bifidobacterium breve, Bifidobacterium catenulatum, Bifidobacterium denticolens, Bifidobacterium dentium, Bifidobacterium gallicum, Bifidobacterium infantis, Bifidobacterium inopinatum, Bifidobacterium longum, Bifidobacterium pseudocatenulatum, Bifidobacterium lactis, Bifidobacterium minimum, Bifidobacterium subtile, Bifidobacterium thermacidophilum, Bifidobacterium animalis, Bifidobacterium asteroids, Bifidobacterium boum, Bifidobacterium choerinum, Bifidobacterium coryneforme, Bifidobacterium cuniculi, Bifidobacterium gallinarum, Bifidobacterium indicum, Bifidobacterium magnum, Bifidobacterium merycicum, Bifidobacterium pseudolongum subsp. Pseudolongum, Bifidobacterium pseudolongum subsp. Globosum, Bifidobacterium pullorum, Bifidobacterium ruminantium, Bifidobacterium saeculare, Bifidobacterium suis, Bifidobacterium thermophilum or yeast cells chosen from Saccharomyces cerevisiae, Saccharomyces carlsbergensis, Saccharomyces chevalieri, Saccharomyces delbrueckii, Saccharomyces exiguous, Saccharomyces fermentati, Saccharomyces logos, Saccharomyces mellis, Saccharomyces oviformis, Saccharomyces rosei, Saccharomyces rouxii, Saccharomyces sake, Saccharomyces uvarum, Saccharomyces willianus, Saccharomyces sp., Schizosaccharomyces octosporus, Schizosaccharomyces pombe, Sporobolomyces roseus, Torulopsis candida, Torulopsis famta, Torulopsis globosa, Torulopsis inconspicua, Trichosporon behrendii, Trichosporon capitatum, Trichosporon cutaneum, Wickerhamia fluoresens, Candida arborea, Candida krusei, Candida lambica, Candida lipolytica, Candida parapsilosis, Candida pulcherrima, Candida rugousa, Candida tropicalis, Candida utilis, Crebrothecium ashbyii, Geotrichum candidum, Hansenula anomala, Hansenula arabitolgens, Hansenula jadinii, Hansenula satumus, Hansenula schneggii, Hansenula subpelliculosa, Kloeckera apiculata, Lipomyces starkeyi, Pichia farinosa, Pichia membranaefaciens, Rhodosporidium toruloides, Rhodotorula glutinis, Rhodotorula minuta, Rhodotorula rubar, Rhodotorula aurantiaca, Saccharomycodes ludwigii, and Saccharomycodes sinenses. For instance, the yeast cells can be of the strain Saccharomyces cerevisiae Hansen AS2.375, AS2.501, AS2.502, AS2.503, AS2.504, AS2.535, AS2.558, AS2.560, AS2.561, AS2.562, or IFFI1048; or Saccharomyces carlsbergensis Hansen AS2.420, or AS2.444. The preferred bacteria used in accordance with the present invention are the feruloyl esterase producing Lactobacilli cells exhibiting the highest levels of FAE activity, which are Lactobacillus fermentum 11976.

In accordance with the present invention, the microcapsules are made of a material chosen from Alginate-Poly-L-lysine-Alginate [APA], Alginate-Chitosan [AC], Alginate-Chitosan-Polyethylene glycol (PEG)-Poly-L-lysine (PLL)-Alginate [ACPPA], Alginate -Poly-L-lysine-PEG-Alginate [APPA], Alginate-Chitosan-PEG [ACP], Alginate-Poly-L-lysine-Pectinate-Poly-L-lysine-Alginate [APPPA], Genipin cross-linked alginate-chitosan (GCAC). The preferred material for the conception of the microcapsules used with the present invention is alginate-poly-L-lysine-alginate (APA).

In accordance with the present invention, the suitable carrier for the suspension of microcapsules is chosen from sterile normal saline and a solution of saline and MRS broth. The preferred carrier used with the present invention is sterile normal saline, health food, health beverage, dairy product or fermented dairy product.

### Materials and Method

### 1. Screening and Selection of an Appropriate Highly FAE Active Isogenic Natural Lactobacillus Strain:

The production of FAE by Lactobacilli is detected in an agar-plate assay. The assay involves the substitution of the main carbon source (glucose) in DeMan, Rogosa, and Sharpe (MRS) agar (Difco, USA) pH 6.5 with 0.3 ml sterile ethyl ferulate (10% w/v in dimethylformamide) at the plate-pouring stage. This supplement is immediately mixed, by swirling, with the agar medium to ensure a homogeneous distribution (a cloudy haze) throughout the plate. Sterile filter disks are impregnated in a 20 h MRS-ethyl ferulate broth culture of the test strain during growth, and placed on MRS-ethyl ferulate agar plates, and incubated for a maximum of 3 days at 30° C. The formation of a clearing zone around the disks indicates feruloyl esterase production. To confirm release of FA, cleared agar samples are extracted three times with ethyl acetate after 1 h soaking in diluted HCl (pH 1•5). The combined organic phases are evaporated under reduced pressure, and redissolved in methanol/water (50/50, v/v) before HPLC analysis (see below). For each set of incubation conditions, samples from uninoculated (hazy) agars are treated similarly and used as controls.

### 2. Microorganisms Used and Their Growth Conditions

Screened natural FAE isogenic bacteria L. farciminis, L. reuteri, L. fermentum 11976 and L. fermentum 14932 are used for in vitro and in vivo studies. These bacteria naturally exhibit high levels of FAE activity. Using cryovials, stock cultures are maintained at −86° C. Bacteria are serially cultivated in (MRS) broth (Difco, USA) followed by serial passages in MRS-EFA broth (MRS supplemented with 1% ethyl ferulate w/v in dimethylformamide) at 37° C. for 20 h in microaerophilic conditions (5% CO2).

### 3. Membrane for Making Artificial Cell Containing Live Bacterial Cells for Oral Delivery:

The Alginate-poly-l-lysine-alginate microcapsule (APA) membrane previously described for the delivery of live bacterial cells is used (Prakash, S. & Jones, M. L. Cell and enzyme compositions for modulating bile acids, cholesterol and triglycerides. 20070116671. 2007). It is be prepared using calcium alginate and poly-l-lysine (PLL), both non-toxic materials. In the APA membrane microcapsule, alginate forms the core and matrix for the cell and PLL binds to the alginate core. Binding of PLL to alginate is the result of numerous long-chain alkyl-amino groups within PLL that extend from the polyamide backbone in a number of directions and interact with various alginate molecules. The resulting cross-linkage produces a stable complex membrane that reduces the porosity of the alginate membrane and forms an immunoprotective barrier. The proposed APA microcapsules are known to have a pore size with an upper permeability limit of 60-70 kDa and the FAE enzyme is known to have a molecular weight of 33-36 kDa. This implies that the FAE enzyme released by the Lactobacillus cells could easily diffuse outside the polymeric membrane of the APA microcapsules. The APA microcapsule has been used successfully to limit the major problem of immuno-rejection related to the use of live cells for therapy and in delivery of live bacterial cells. Other suitable membranes will be readily apparent.

### 4. Method for Making and Storing Artificial Cell Microcapsules:

For this, automated Inotech Encapsulator is used. This equipment is based on the principle that a laminar liquid jet is broken into equally sized droplets by a superimposed vibration and can produce large amount of superior quality microcapsules. Briefly, APA microcapsules are produced by the immobilization of individual cells in an alginate droplet that is then hardened by gelation in a Ca²⁺-rich solution. After gelation in calcium chloride, the beads are washed in a PLL solution to form a membrane that is permselective and immunoprotective. Lastly, the capsules are washed and suspended in a solution of alginate to bind all positively charged PLL residues still present at the capsule surface. The APA system employing polyelectrolyte complexation has proven advantageous, as its aqueous-based, relatively mild encapsulation conditions do not compromise cell viability. The formed microcapsules are stored at 4° C. in a 90:10 (vol/vol) solution of saline and MRS broth and used for the experiments.

### 5. Microcapsule Characterization:

All the microcapsule membrane formulations are characterized for their physiological, biochemical, and functional properties in vitro. Specifically, the following studies are performed.

a) Microcapsule morphology study: Microcapsule morphology is determined using optical microscopy. Further, a comparative study of the characteristics of microcapsules and swelling dynamics under varied pH and other conditions found in GI tract are assessed. Facilities and expertise for these studies exist at the laboratory.

b) Microcapsule stability studies in computer controlled GI model: To test the microcapsule formulation, a dynamic simulated human GI tract model using five reactor vessels is used. Each of the five reactor vessels represents distinct parts of the human GI tract in the following order (reactors 1-5): stomach, small intestine, ascending colon, transverse colon and descending colon. Each reactor vessel has eight ports: for input and output of the medium, sampling of liquid phase, gas, pH electrode, pH control (acid and base), and for flushing of head space. The pH of the reactors 2, 3, 4 and 5 is controlled between 6.5 and 7.0, 5.5 and 6.0, 6.0 and 6.4, 6.4 and 6.8 respectively using 0.5M NaOH, and 0.5M HCl. The pH in reactor 1 is kept at 2.0-2.5 by adding HCl in order to simulate the acidic effects of the stomach. The GI model is fed three times a day with feed medium comprised of glucose 0.4 g/day, arabinogalactan 1 g/day, pectin 2 g/day, xylan 1 g/day, starch 3 g/day, yeast extract 3g/day, peptone 1 g/day, mucin 4 g/day, and cysteine 0.5 g/day as previously described. All experiments are carried out at body temperature (37° C.). All physiological and biochemical parameters of the model, including transfer of content from one vessel to other, are computer controlled using a LabView 6i software. This in vitro system closely mimics the in vivo conditions with regard to pH, temperature, bacteria, types of enzymes, enzymatic activity, volume, stirring, and possible food particles. The computer controlled dynamic GI model is fully functional.

As mentioned above, this dynamic in vitro GI tract model mimics the various stages and conditions of the human intestinal tract. The fates of formulation after oral administration are often studied in simple, static models; a dynamic model provides more realistic results. This model supplies realistic information about the stability, release and absorption of various compounds during passage through the GI tract and allows the applicants to optimize the microcapsule formulations with regards to cellular viability, capsule integrity and other parameters under in vivo conditions of pH, bacteria, enzymes, enzymatic activity, volume, food stuffs and active micro flora.

### 6. Microencapsulated Bacterial Viability Studies in Various GI Conditions In Vitro:

In vitro studies are performed to evaluate the susceptibility of microencapsulated bacteria to GI tract conditions. Specifically, all pH that orally delivered microencapsulated bacteria are likely to confront are evaluated. Acidic conditions encountered in the stomach and pancreatic juices, bile enhanced conditions encountered in the duodenum are of particular interest. All experiments are carried out initially in a 250-ml flask at 37° C. and 100 rpm shaking, and later in the dynamic in vitro GI model.

Evaluation of the microencapsulated Lactobacillus cells for FAE activity and bound FA de-esterification in vitro The experiment is carried out in “simulated” GI fluids in flask conditions. A previously described method is modified and used to investigate the FAE activity of microencapsulated Lactobacilli cells in the flask. The assay is based on the measurement of FA released from the substrate. One volume of encapsulated FAE producing Lactobacilli cells is mixed with 3 volumes of 1.33 mM ethyl-ferulate in “simulated” media, pH 6.5. Both are preheated to 37° C. before mixing. The final mixture is incubated at the same temperature. Blanks containing the ethyl-ferulate in simulated GI fluid media are incubated as controls. A second blank containing the encapsulated Lactobacilli cells is also incubated to check for presence of FA in the media sample. At various time intervals, aliquots of the reaction mixture are withdrawn and mixed with 0.35 M H2SO4 to stop the reaction. This is followed by the addition of 1.0 mM benzoic acid as internal standard and 0.7 M NaOH. The solution is mixed by vortexing, passed through a 0.45-μm syringe filter, and analyzed. A high pressure liquid chromatography (HPLC) procedure will be used to determine released FA. The HPLC system is made up of two ProStar 210/215 solvent delivery modules, a ProStar 320 UV/Vis Detector, a ProStar 410 AutoSampler, and the Star LC Workstatin Version 6.0 software will be used.

Analyses are performed on a reversed-phase C-18 column: LiChrosorb RP-18, 5 μm, 250×4.6 mm from Richard Scientific (Novato, Calif., USA). An isocratic elution with water:acetic acid:1-butanol (350:1:7 v/v) as the mobile phase is used at a flow rate of 1.0 mL/minute at ambient temperature. An injection loop of 20 μL is used, and the detection wavelength set to 279 nm. The system is equilibrated by the mobile phase. External standards of trans-FA (Sigma, USA) are accurately weighed and dissolved in minimal amount of ethanol and water to give serial concentrations. Identification of the compounds is confirmed by comparing retention times and absorption spectra to those of standard materials. Quantification is accomplished using calibration of the external standards.

### 7. Experimental Animal Model and In Vivo Experimental Procedure:

The in vivo animal studies evaluate the suitability of the microcapsule ormulation and their pre-clinical liver lipid lowering efficacy in experimental animals by oral administrations. Studies optimize dosage, their safety and toxicological evaluations. For the in vivo experiments, to evaluate therapy formulation's efficacy, several animal models are available. However, we have elected to use golden Syrian hamster model. This is because in this model fatty liver conditions can be induced easily by diet. In addition, this model is known to be very similar to human hepatobilliary circulation compared to ob/ob mice or other animal model used by others. Furthermore, these animal can be made hyperlipidemic by diet supplements and are known to develop diabetes when on high cholesterol diet, and always shows better AI profile.

Indeed this animal model has been for formulation evaluations by others. For the experiment, animals are purchased from BioBreeders USA) and used for the experiments.

For the in vivo animal study, male golden Syrian hamsters (strain Bio F1B, BioBreeders USA), aged 4-6 weeks and weighing ˜70 g at reception, are placed two per cage and are acclimatized to the facility (sterile room with controlled temperature (22-24° C.) and inversed, alternating light and dark cycles). Food and water are provided ad libitum.

A total of 7 groups, each consisting of 12 young male Golden Syrian Hamsters, are employed for the experiment. All 84 hamsters are fed a normal diet (Rodent Chow 5001) for 2 weeks along with reversed light and dark cycles and saline gavage to acclimatize them to their new environment. After the acclimatisation period, saphenous vein blood collection is performed on the hamsters and the serum analysed for total lipids (cholesterol+triglycerides). Based on these values the group of hamsters are split into the test and control groups based on a semi-block design. The groups are as follows:

(A) Normal hypercholesterolemic treatment group #1

(B) Normal hypercholesterolemic treatment group #2

(C) Normal hypercholesteroemic control group

(D) Fatty Liver induced normal hypercholesterolemic treatment group

(E) Fatty Liver induced normal hypercholesterolemic control group

(F) Fatty Liver induced diabetic hypercholesterolemic treatment group

(G) Fatty Liver induced diabetic hypercholesterolemic control group

Groups A-C are fed a high cholesterol diet throughout the experiment and given microcapsule treatment (two groups) or control.

Groups D-G are all induced for fatty liver and only the groups (F) and (G) for diabetes as below:

For the induction of diabetes: Food is withheld from 24 hamsters for 2 h during their dark cycle. They are given an intra-peritoneal (IP) injection of streptozotocin (STZ) (Sigma Chemical, St. Louis, Mo.), 50 mg/kg B.W. dissolved in a citric acid buffer (pH 4.5) for three consecutive days. Immediately on injection, the animals in all cages are provided with a 5% Glucose solution to overcome the drug induced hypoglycemia. Cages housing animals induced for diabetes are not changed for 1 week owing to biohazardous nature of STZ. Eight to ten days later, glucose concentrations in blood samples are measured. Animals with blood glucose levels of ≧250 mg/dl (13.88 mM) are selected to continue the dietary and formulation treatments.

For the induction of fatty liver: Once diabetes has been induced, all animals in all four groups are fed a methionine deficient-choline devoid, synthetic diet containing 0.05% cholesterol and 6% saturated fats in addition to other essential vitamins, minerals, nutrients, etc. (from Land O' Lakes Purina Feed, LLC, Test Diet Formulation 5D4F) for 10 days—[MDCD]

After 10 days of feeding the MDCD diet, 1-2 hamsters in each group are euthanized to determine the development and progression of fatty liver disease in the animals. Twelve animals per group are initially selected to provide adequate numbers of statistical analysis of the study data allowing that not all 24 animals will develop elevated serum glucose levels.

For the remainder of the experiment (15 weeks) groups D-G are fed a methionine adequate-choline deficient non-purified diet containing 0.05% cholesterol and 6% saturated fats in addition to other essential vitamins, minerals, nutrients, etc. (from Land O' Lakes Purina Feed, LLC, Test Diet Formulation 5D4E). [MACD].

One group of diabetic hamsters [Group G], used as a positive diabetic control group (n=10), receive the MACD diet with sham (empty) microcapsule intervention. The second diabetic group [Group F] receive the MACD diet supplemented with encapsulated lactobacillus formulation treatment (n=10). The other 2 groups [Groups D and E] comprise of non-diabetic hamsters who receive the MACD diet and are used as non-diabetic controls receiving sham (empty) microcapsules and encapsulated lactobacillus formulation treatment (n=10) respectively.

Subsequently after treatment period is complete, a follow up commences, whence all the animal groups are fed high lipid diet for another 4 weeks. In these animals, we evaluate effectiveness of treatment in maintaining low levels of serum and hepatic lipid.

Throughout the experiment, weight gain and food consumed in each group are monitored weekly. Venous blood samples are collected biweekly (preceded by a 12-14 hour fast). The samples are centrifuged and assayed for total cholesterol (TC), HDL levels, LDL levels, C-reactive protein levels, plasma triglycerides (TG), ALT, AST, serum glucose as well as for other relevant molecules using a Hitachi 911 clinical chemistry analyzer and HPLC system available in our research laboratory. We also collect fecal samples of these animals on a weekly basis and analyze for bile and sterols. Furthermore, when the studies are completed, we sacrifice animals using standard procedure and collect the their tissue samples (e.g. livers, aortic arches, gal bladder, and others) for further analysis. Obtained liver samples are analyzed for hepatic lipid levels, C-reactive protein, HMG-CoA reductase, ACAT, and aminotransferases enzymes (AST and ALT) levels in all groups of animals. We also perform histological analysis of microtomed liver samples to show efficacy of the treatment.

### 8. Formulation Toxicology Evaluations:

These formulations are known not to exert any toxic effects, as Lactobacilli, and Bacillus calls are commonly found in food such as in yoghurt and are commonly found in human gut and materials used in making capsules. However, safety data are essential. We evaluate microcapsule formulation safety/toxicity in vivo in experimental animals. For this, we deliver suitable amount of formulation orally and evaluate animal toxicity in animals. On these animals we observe: 1.) Survival curves for animals receiving different doses of microcapsule formulations, 2) Appetite/General Health (body weight, feed/water consumption), 3) Ocular Observations (corneal opacities, nystagmus, alopecia, pupillary changes, blindness, discharge, conjunctivitis, weight loss as a sign of systemic toxicity/cachexia, as well as weight gain), 4) Integument (erythema, haircoat condition, status of hydration, pruritus), 5) Equilibrium (unsteadiness on legs, coordination of legs, abnormal reflexes), 6) Muscular disturbances (generalized tremors, lip drooping, paralysis), 7) Cardiovascular (heart rate),8) Behavior (anxious, restless, aggression, sedated, shaking head), 9) Respiratory (respiratory sound), 10) Hematology (complete blood count, platelet count, hemoglobin), 11) Blood and Serum Chemistries (glucose, creatinine, sodium, potassium, total protein, albumin, BUN and pH), 12) Urinalysis (visual observations, pH, protein, bilirubin, ketones), and 13) Fecal Examination (quantity, color, blood or other signs). We also perform histological analysis of tissue sections of the duodenum, liver, spleen, kidney, heart and lung of treated animals.

### 9. Statistical Analysis

Most of the outcome variables evaluated are continuous variables and are analyzed descriptively in terms of their means (standard errors). Differences in the therapeutic benefits between the study formulation and result of other approaches for the same therapy are analyzed using Student's t-tests with statistical significance estimated at the 5% level (p value less than 0.05). Differences between 2 or more groups are evaluated using Analysis of variance (ANOVA) techniques. Repeated measures data are compared using Analysis of Covariance (ANCOVA) for repeated measures. Appropriate transformation of study variables is done when required.

The present invention will be more readily understood by referring to the following examples, which are given to illustrate the invention rather than to limit its scope.

### Example I

### Screening of Lactobacilli for Enzyme Feruloyl Esterase (FAE) Activity for Use in Oral Formulation

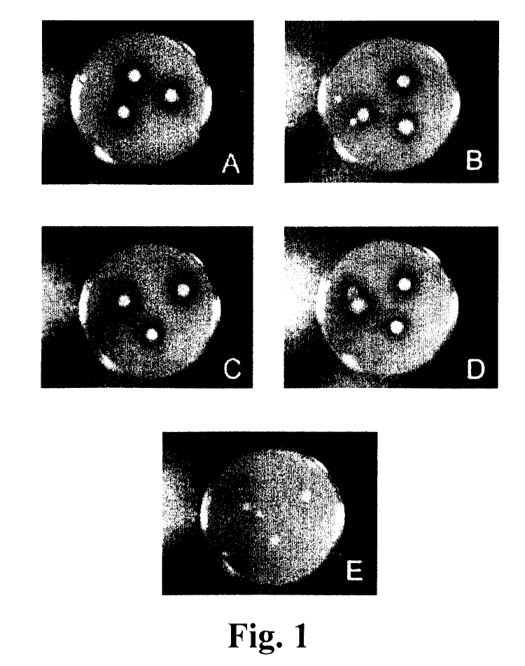

Qualitative FAE activity of the Lactobacilli was evaluated in an agar-plate assay. The assay involves the substitution of the main carbon source (glucose) in MRS agar with ethyl ferulate (10% w/v in dimethylformamide). This supplement ensures a homogeneous cloudy haze throughout the plate. The ability of each strain to de-esterify ethyl ferulate was assessed. The formation of a clearing zone around the disks (impregnated with bacteria) indicates feruloyl esterase production (FIG. 1). Lactobacillus reuteri, Lactobacillus farciminis, Lactobacillus fermentum 11976, and Lactobacillus fermentum 14932 were tested for FAE activity. A negative control was established with sterile media in flasks and sterile filter paper disks on plate. Of the 4 strains screened for FAE activity on plates, all 4 returned positive results, with clearance zones differing in size (Table 1 below). L. farciminis was found to exhibit the greatest ethyl ferulate de-esterification activity and thus showed the most significant zones of clearance with an average diameter of 14.2 mm indicating highest FAE activity. Based on the zones of precipitation formed, the 4 strains displaying the larger clearance zones were selected for microencapsulation and further study.

TABLE 1
Culture tolerance to ethyl ferulate and FAE activity as
detected by the plate assay method ^{a} Dc, diameter
of the clearance zone. Each value represents the average
from three measurements ± standard deviation.

| Filter paper disk | | | |
|---|---|---|---|
| impregnated with | Growth in MRS-EFA broth | Dc^{a} | |
| Lactobacillus fermentum | Average | 12.2 ± 1 | mm |
| 11976 | | | |
| Lactobacillus fermentum | Average | 12.3 ± 0.6 | mm |
| 14932 | | | |
| Lactobacillus reuteri | Average | 12.7 ± 0.6 | mm |
| Lactobacillus farciminis | Average | 14.2 ± 0.8 | mm |
| No bacteria control | None | 0.00 | mm |

### Example II

### In Vitro Stability of APA Polymeric Microcapsule and Bacterial Viability Under GI Conditions for Oral Delivery

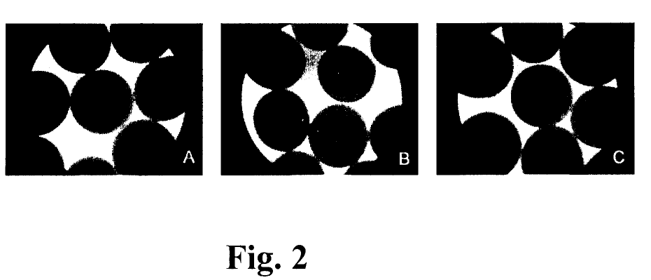

The viability and sensitivity of the encapsulated bacteria to Simulated Gastric Fluid (SGF), acidic conditions, Simulated Intestinal Fluid (SIF) and stability of APA capsules to mechanical shear was evaluated. To simulate the stomach conditions, microcapsules were incubated at 37° C. in SGF with mechanical shaking (150 rpm), followed by 10 hours in SIF. During simulated gastric and intestinal transit, the integrity of over 90% of APA microcapsules was retained (FIG. 2).

On exposure to synthetic gastric fluids and mechanical shaking, the microencapsulated bacteria showed a slight decrease in viability as compared to untreated microcapsules, however, the viability was even so, adequate for probiotic usage purposes. L. farciminis and L. reuteri microencapsulated cells showed a greater survival than those of L. fermentum strains after gastric treatment (Table 2 below). When the Lactobacilli cells entrapped in beads were exposed to simulated gastric juices, the death rate of the cells in the beads increased proportionally with an increase in time of exposure to the SGF. Later, after exposure to SIF, the bacteria were found to have multiplied and the number of viable cells increased, as a consequence of metabolism in SIF. The change in media pH in the SIF over the duration of the experiment illustrates the metabolic activity of the encapsulated bacteria (Table 2).

TABLE 2
Viability of microencapsulated Lactobacilli cells after
refrigerated storage, gastric treatment and intestinal
treatment and effect of bacteria on pH of surrounding media

| | | | Initial | |
|---|---|---|---|---|
| Experimental | Bacterial | Viability | media | Final |
| Stage | Strain | (cfu/mL) | pH | media pH |
| Untreated, | L. reuteri | 4.18 × 10¹⁰ | | |
| Refrigerated Storage | | | | |
| Gastric Treatment | | 5.65 × 10⁹ | 2.0 | |
| Gastric + Intestinal | | 1.3 × 10¹¹ | 7.19 | 3.51 |
| Treatment | | | | |
| Untreated, | L. farciminis | 5.88 × 10¹⁰ | | |
| Refrigerated Storage | | | | |
| Gastric Treatment | | 1.47 × 10⁹ | 2.0 | |
| Gastric + Intestinal | | 6.0 × 10¹⁰ | 7.19 | 3.46 |
| Treatment | | | | |
| Untreated, | L. fermentum | 3.24 × 10¹² | | |
| Refrigerated Storage | 11976 | | | |
| Gastric Treatment | | 7.67 × 10¹⁰ | 2.0 | |
| Gastric + Intestinal | | 2.05 × 10¹² | 7.19 | 3.72 |
| Treatment | | | | |
| Untreated, | L. fermentum | 6.85 × 10¹² | | |
| Refrigerated Storage | 14932 | | | |
| Gastric Treatment | | 7.69 × 10¹⁰ | 2.0 | |
| Gastric + Intestinal | | 1.69 × 10¹² | 7.19 | 3.85 |
| Treatment | | | | |

### Example III

### “Real-Time” Bacteria FA Release Assay: HPLC Analysis

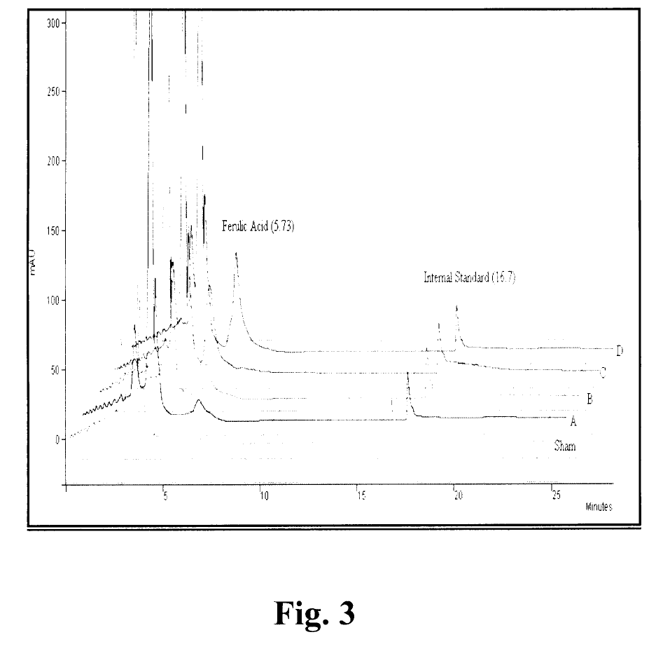

Quantitative measurement of ferulic acid released from ethyl ferulate by the FAE activity of microencapsulated L. fermentum 11976 was carried out by high-performance liquid chromatography (HPLC). FIG. 3 shows the HPLC chromatogram depicting the de-esterification of ethyl ferulate in 10 hours by gastric stressed Lactobacillus microcapsules. The activity of Lactobacillus microcapsules was compared with control empty microcapsules.

L. fermentum 11976 microcapsules de-esterified ethyl ferulate at a significantly greater rate (9.12 nmol FA released/g CWW/h) than the other encapsulated Lactobacillus strains to release ferulic acid. Furthermore, both encapsulated L. fermentum strains showed higher FAE activity than encapsulated L. farciminis or L. reuteri (Table 3 below). As seen in Table 3, the average amount of ferulic acid liberated from ethyl ferulate over 10 hours was 7.40 nmol FA released/g CWW/h for L. fermentum 14932 microcapsules, 3.16 nmol FA released/g CWW/h for L. reuteri microcapsules and 1.78 nmol FA released/g CWW/h for L. farciminis microcapsules.

TABLE 3
FAE activity (microgram FA released/g capsule wet weight/h) of
microencapsulated Lactobacilli cells from 1.33 mM ethyl-ferulate

| | Feruloyl esterase activity |
|---|---|
| | (μg ferulic acid released/g capsule |
| Bacterial strain | wet weight (CWW)/h) |
| Microencapsulated L. farciminis | 12.24 |
| Microencapsulated L. reuteri | 6.88 |
| Microencapsulated L. fermentum | 35.32 |
| 11976 | |
| Microencapsulated L. fermentum | 28.67 |
| 14932 | |

### Example IV

### In Vivo Evaluation of Formulation Efficacy in Experimental Hamsters

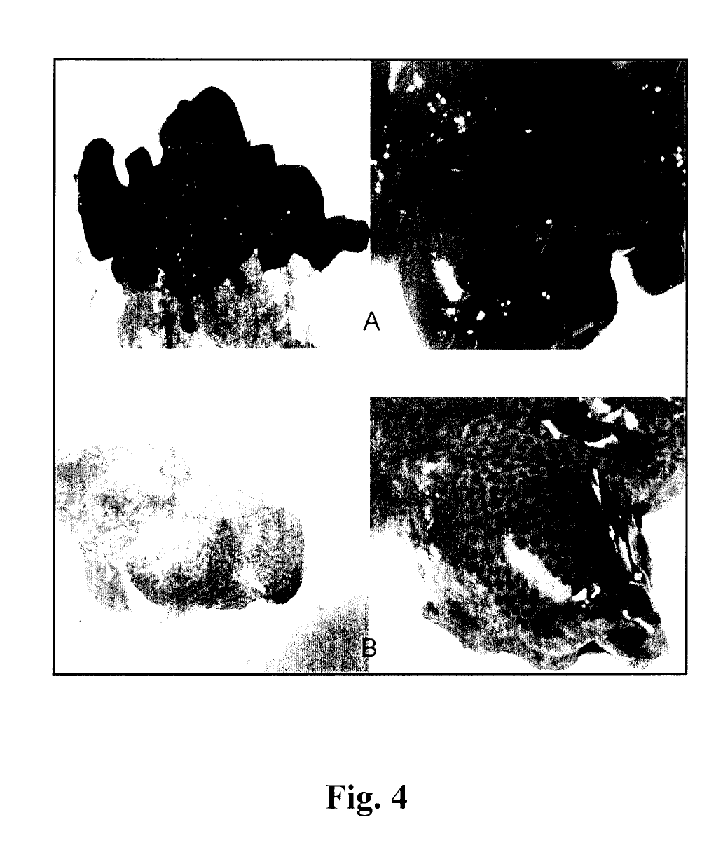

Analysis of the animal model (F1B male Golden Syrian hamsters, Biobreeders, USA) and in vivo studies of the feasibility of the formulation for the proposed approach for NAFLD therapy were carried out. The in vivo animal studies showed the suitability of the microcapsule formulation for oral delivery of Lactobacillus cells, the efficacy of such encapsulated bacteria in improving the hepatic lipid profile, and providing data showing their safety in a diet-induced fatty liver, hyperlipidemic, atherosclerotic and diabetic animal model, the male golden Syrian hamster model. In particular, the Bio F1B strain (BioBreeders USA) male golden Syrian hamster was employed because of its characteristic phenotype which promotes diet-induced hyperlipidemia and atherosclerotic lesion formation. For the in vivo animal study, male golden Syrian hamsters (strain Bio F1B, BioBreeders USA), aged 4-6 weeks and weighing ˜70 g at reception, were fed with a non-purified hypercholesterolemic, hyperlipidemic diet consisting of wheat bran enriched chow supplemented with 5% coconut oil and 0.05% cholesterol by weight. Control animals were fed with a regular chow based diet. The study in our lab and studies elsewhere showed that experimental hamsters fed a HC diet, not unexpectedly, gained weight and showed an increased lipid profile and developed fatty livers (FIG. 4). Control hamsters fed a regular chow diet did not show fatty deposits in liver (FIGS. 4A and 4B). On the other hand, the test animals fed a high lipid diet showed increased hepatic fat depositions resulting in fatty liver (FIGS. 4C and 4D).

### ii) Efficacy of Formulation in Lowering Fatty Deposits in Livers of Golden Syrian Hamsters

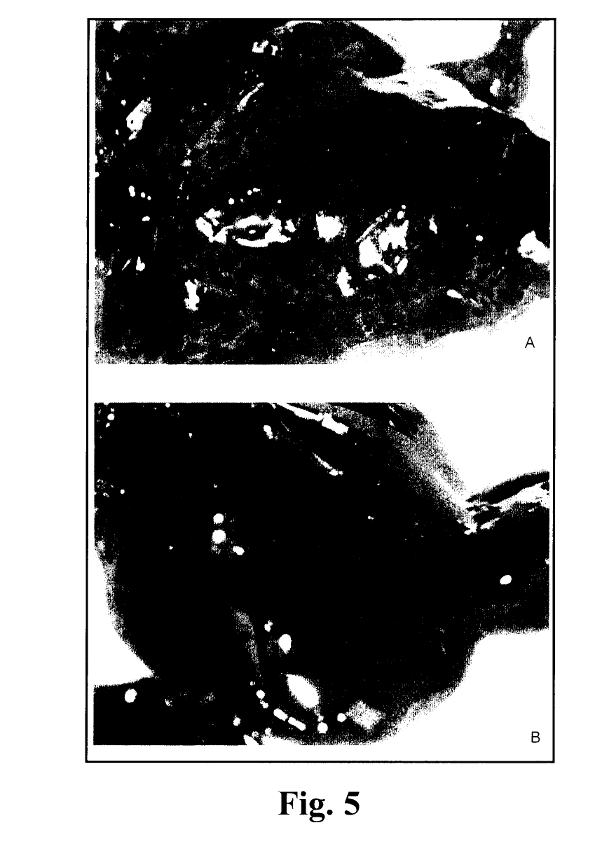

For the in vivo animal study, male golden Syrian hamsters (strain Bio F1B, BioBreeders USA), aged 4-6 weeks and weighing ˜70 g at reception, were fed a chow based diet for 2 weeks in order to acclimatize them to the facility and inversed, alternating light and dark cycles). Food and water was provided ad libitum. Control microcapsules or microencapsulated bacterial cells were orally force fed to test hamsters daily using stainless steel gavage. After a 8 week treatment period, the end point of the short term experiment, and a 20 week treatment period, the end point of the long term experiment, the test animals were euthanized using carbon dioxide gas and their livers removed. The livers of the control animals were shown to have extensive fatty deposits in the vasculature (FIG. 5A). The test group of animals was fed microencapsulated Lactobacillus cells and at the end point, the livers of this group were found to have reduced lipid deposits in the hepatic vasculature as compared to non-treated controls (FIG. 5B). This demonstrates the efficacy of the oral polymeric membrane Lactobacillus formulation in NAFLD therapy.

### Example V

### Cholesterol Lowering Efficacy of the Oral Formulation in a Golden Syrian Hamster

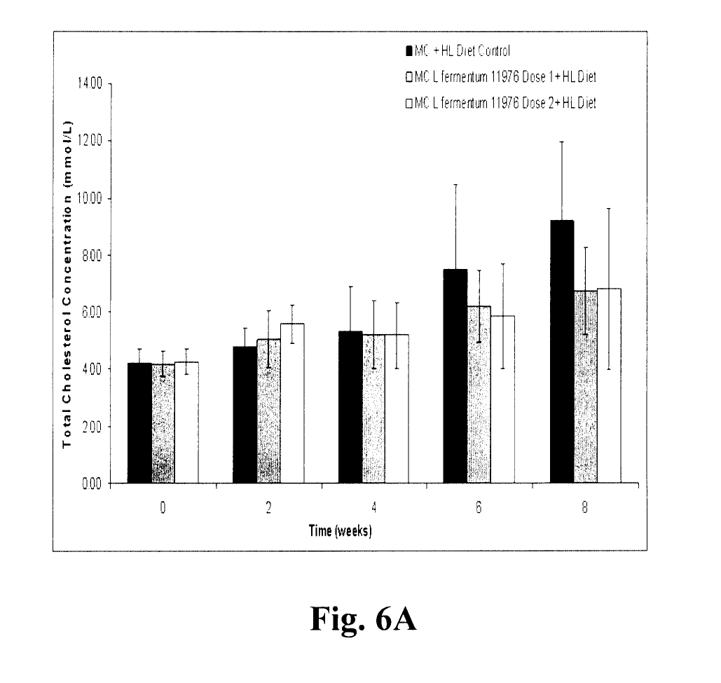

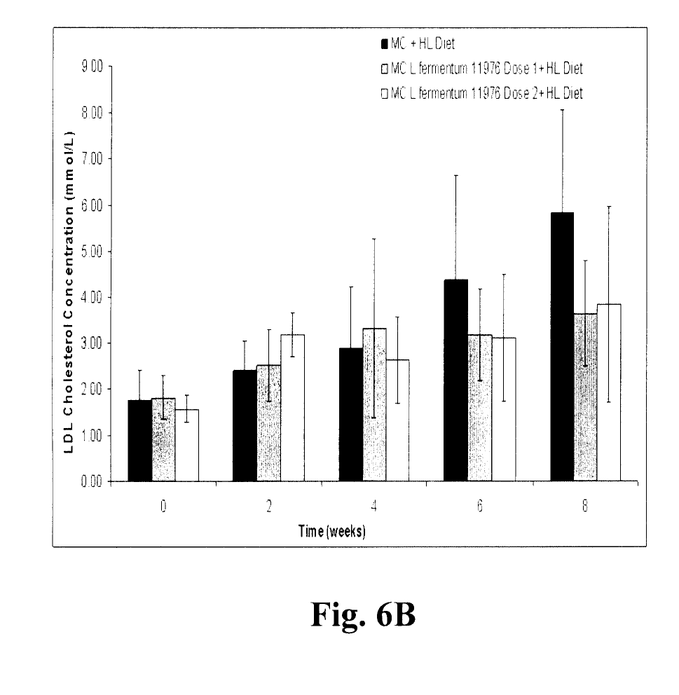

The microencapsulated L. fermentum 11976 cells formulation was tested at 2 different dosages in the animal model for eight weeks for lipid and blood glucose reduction when compared to control animals. For the test, empty microcapsules or microcapsules containing bacterial cells were orally force fed to test hamsters daily using stainless steel gavage. Throughout the experiment, weight gain and food consumed in each group was monitored weekly. Venous blood samples were collected biweekly (preceded by a 12-14 hour fast) for 8 weeks. The samples were centrifuged, and assayed for total cholesterol (TC), HDL levels, LDL levels, using a Hitachi 911 clinical chemistry analyzer available in our research laboratory. Our results show total cholesterol and LDL cholesterol stabilized over 4 weeks, after which in the 6^{th} and 8^{th} weeks a significant decrease (p<0.05) in the levels of serum total (FIG. 6A) and serum LDL cholesterol (FIG. 6B) was observed. Serum total cholesterol was found to decrease by 27% and serum LDL-cholesterol was observed to decrease by 37.37% at the eight week of treatment as compared to sham-treated controls.

### Example VI

### Triglyceride Lowering Efficacy of the Oral Formulation in a Golden Syrian Hamster

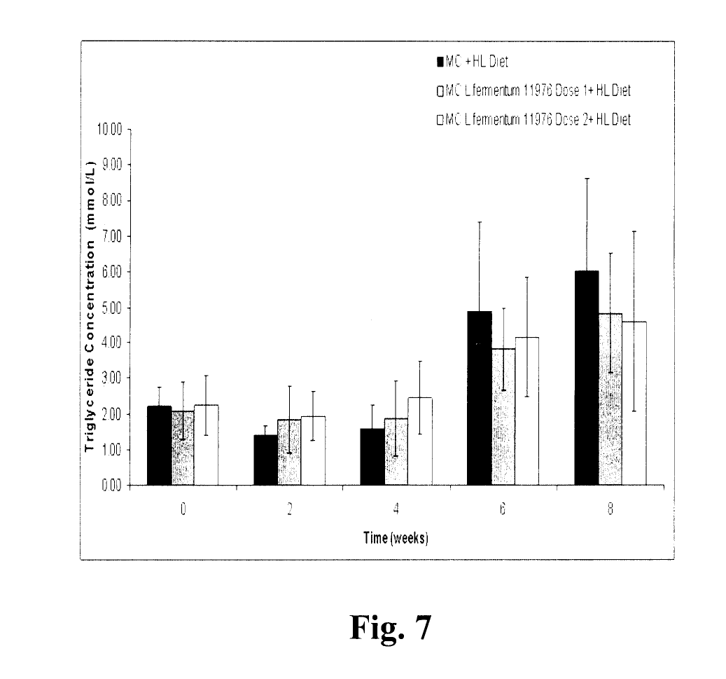

Increased cardiovascular risk reflects an increase in circulating cholesterol and triglycerides. Thus it is beneficial to reduce the concentrations of cholesterol and triglycerides. The treated and control hamsters were maintained under equivalent nutritional and environmental conditions. Treatment over eight weeks in test animals reduced triglycerides by 23.42% in the hamsters fed microencapsulated L. fermentum 11976 formulation but because of the large variation the difference did not reach statistical significance (FIG. 7). In sharp contrast, triglycerides in control hamsters exceeded the microcapsule formulation treated group triglycerides.

### Example VII

### Efficacy of the Oral Formulation on Atherogenic Index in a Golden Syrian Hamster

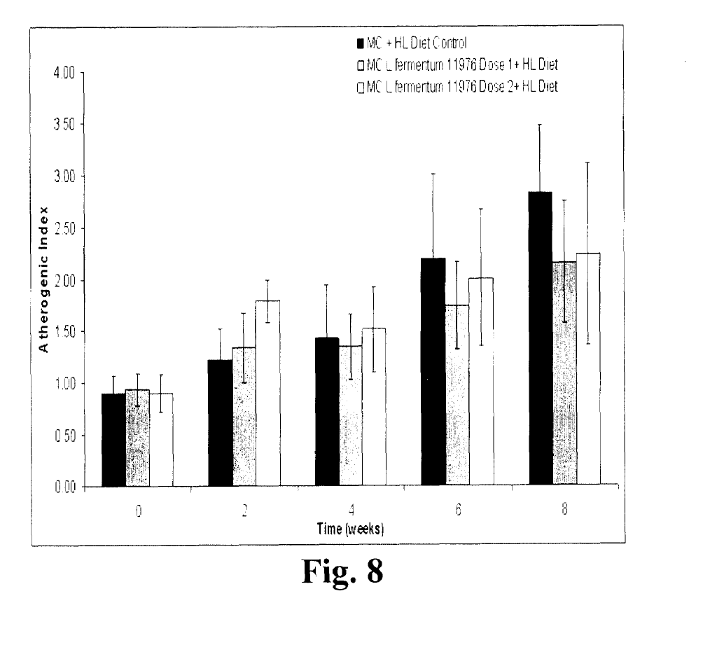

One major risk for coronary heart disease is elevated serum cholesterol levels and lower density lipoproteins are rendered atherogenic by oxidation in the wall of the artery. The one-to-two rule applies, which states that a 1% reduction of serum cholesterol causes a 2% reduction of the risk for coronary artery disease. Chronic exposure to fat and cholesterol leads to inflammation of the liver that precedes lesion formation in the aorta. NAFLD is known to be associated with carotid atherosclerosis. Hence an estimate of the atherogenic index in the hamster is a valuable indicator of liver inflammation. Over the treatment period of eight weeks, the test hamsters which were orally fed the microencapsulated L. fermetum 11976 formulation showed a significant (p<0.05) decrease in the atherogenic index when compared to the control group (FIG. 8).

### Example VIII

### Efficacy of the Oral Formulation for Lowering Blood Glucose

Following a 2 week acclimatization of basal diet and saline gavage, animals were sorted into the control and the treatment groups based on basal serum TC concentrations to start the experimental period. During the 20 week experimental treatment period, all animals were fed a hypercholesterolemic diet. Compared to the baseline level, serum TC levels were quite elevated in all groups of animals after 20 weeks on the test diet. As other short-term studies have shown, this elevation likely resulted from the dietary cholesterol ingested.

The microencapsulated LF11976 treatment group showed a significant reduction in serum TC (11.95%; p=0.0065), LDL cholesterol concentrations (18.07%; p=0.0046) and the atherogenic index (12.33%; p=0.0034) with respect to control [FIGS. 10(a), 2(c) and 2(e)] at the end of the experimental period. The high density lipoprotein (HDL) cholesterol concentration was also found to decrease significantly (3.69%, p=0.0394) when compared to control [FIG. 10(b)]. There was no statistically significant difference in serum triglyceride (TG) levels although it was shown to reduce 2.4% in the microencapsulated LF11976 treatment group relative to the control [FIG. 10(d)]. There was also no significant difference between the body weights of the control (164±16 g) and treatment (167±13 g) groups at the end of the experiment [FIG. 10(f)].

### Example IX

### Effect of Varying Dosage of Microencapsulated LF11976 Treatment on Hypercholesterolemic Hamsters

All hamsters in each group survived for the entire length of the study. All groups gained significant amounts of body weight during the 20-week study. The body weights at the beginning and end of the experiment were 102±9 g and 164±16 g for the control group and 104±8 g and 167±13 g for the low dose group, 105±6 g and 161±13 g for medium dose group and 107±6 g and 149±9 g for high dose group, respectively. At the same time, no significant difference for food consumption between the treatment diets was observed.

Serum TC and LDL cholesterol concentrations were significantly lower in the hamsters fed the low dose (12%; p=0.01 and 18%; p=0.01, respectively), the medium dose (21%; p=0.04 and 28%; p=0.03 respectively) and the high dose (24%; p=0.01 and 26%; p=0.03 respectively) of the microencapsulated LF11976 formulation as compared to the empty microcapsule fed hamsters [FIGS. 11(a) and 11(c)] at the end of 20 weeks. Relative to the sham (empty microcapsules) fed hamsters, serum HDL cholesterol concentrations were also significantly lower (p<0.05) in hamsters fed low, medium and high doses of the microencapsulated LF11976 formulation (4%; p=0.04, 7%; p=0.04 and 9%; p=0.002, respectively) [FIG. 11(b)]. Although not significant, the hamsters fed microencapsulated LF11976 had lower serum TG concentrations compared to the empty microcapsule fed controls [FIG. 11(d)]. Despite the decrease in HDL cholesterol, the animals treated with the low dose of microencapsulated LF11976 formulation were shown to significantly maintain a more optimal atherogenic index (12% decrease; p=0.003) when contrasted with the control hamsters [FIG. 11(e)]. In contrast, the decrease in the atherogenic index of hamsters fed the medium and high doses of encapsulated bacteria, when compared to the control hamsters, was not significant (p>0.05).

To determine whether the formulation differed in effectiveness when administered to animals of differing basal serum cholesterol, hamsters were ranked according to their basal serum TC and the median (4.2 mM) was considered as the cut-off point between low (<4.2 mM) and high (>4.2 mM) serum TC concentrations for each dose of treatment.

In the dose-fed treatment groups with low (<4.2 mM) basal serum TC, serum lipid and lipoprotein concentrations as well as the atherogenic index were most elevated in hamsters gavaged the high dose (12.98 log cfu/mL) of microencapsulated LF11976. Although the TC, HDL, LDL cholesterol, TG concentrations and AI were numerically lower after treatment than controls, the differences were not significant at the 0.05 probability level.

In comparison, the group of animals gavaged the low dose of the formulation (12.51 log cfu/mL) demonstrated significant reduction in serum TC (23% decrease; p=0.004), HDL (6% decrease; p=0.03), LDL (28% decrease; p=0.01), TG (25% decrease; p=0.01), and the AI (22% decrease; p=0.003) after 20 weeks of treatment relative to controls [FIGS. 12(a), 12(b), 12(c), 12(d), 12(e)]. Twenty weeks of treatment with the medium dose of the formulation resulted in no significant reduction in HDL or TG; however, the serum TC, LDL and Al were significantly lowered as compared to untreated controls (45% decrease; p=0.04; 51% decrease; p=0.02; 37% decrease; p=0.04 respectively).

Serum TC, HDL, LDL cholesterol and TG were not significantly different between controls and treatment group hamsters with high (>4.2 mM) basal TC levels, when gavaged with the low and medium doses of the formulation. Interestingly, for hamsters treated with the high dose, a significant lowering response was observed for serum TC (33% decrease; p=0.02), LDL cholesterol (36% decrease; p=0.02) and HDL cholesterol (14% decrease; p=0.01) but not for serum TG [FIGS. 13(a), 13(b), 13(c), 13(d)]. The Al index was shown to decrease numerically with all three dosages of the formulation in hamsters with greater than 4.2 mM basal TC levels; however these reductions were not found to be statistically significant [FIG. 5(e)].

The diet, and thus the cholesterol intakes of all these groups of animals were similar; their body weight gains also did not differ significantly. As such, these were not important factors affecting serum cholesterol response.

### Example X

### Formulation Efficacy in Obesity, Dyslipidaemia, Insulin Resistance (IR) and Type II (Non-Insulin Dependent) Diabetes Mellitus

NAFLD is strongly associated with obesity, dyslipidaemia, insulin resistance (IR) and type II (non-insulin dependent) diabetes mellitus. A most successful strategy to prevent diabetic complications is to prevent hyperglycemia and thereby oxidative stress and increase insulin sensitivity. The changes in levels of blood glucose in hamsters treated with the microcapsule oral L. fermentum 11976 formulation and sham microcapsule control are shown in FIG. 9. Further studies with different dosages of the microencapsulated LF11976 bacteria over a longer term of 20 weeks, showed that elevated glucose levels were significantly decreased as compared to control animals. [Treatment D1: 17% decrease; p=0.0085, Treatment D2: 10% decrease; p=0.0164 and Treatment D3: 13% decrease; p=0.0372] (FIG. 9A). The treatment stabilizes and even decreases blood glucose levels. The effect is more pronounced for the low dose than that of the high doses of microcapsule formulation. The exact mechanism for glucose lowering is not known. This decrease in blood glucose levels is useful to treat diabetes patients.

### Example XI

### Formulation Efficacy in Managing Aminotransferases (Aspartate Transaminase AST and Alanine Transaminase ALT)

Increases in serum aminotransferases (aspartate transaminase AST and alanine transaminase ALT), are the only biochemical indicators of NAFLD. Normal biochemical values have been found in pathologically obese individuals, whose hepatic biopsies indicated progressive liver disease. Also, the AST/ALT ratio can be useful for distinguishing nonalcoholic hepatic steatosis from alcoholic hepatic steatosis, a pathology in which profound anatomopathologic changes in the liver can be caused by abuse of alcohol (ethanol). In alcoholic steatosis the AST/ALT ratio is typically greater than 2 whereas in nonalcoholic steatosis the levels of ALT are higher than those of AST. In our experiment, the values for average ALT and AST after 16 weeks of therapy had decreased numerically by 24% and 19% respectively relative to the control values in hamsters fed a hyperlipidemic, hypercholesterolemic diet (Table 4).

TABLE 4
The data given shows that treatment of NAFLD with
microencapsulated LF11976 therapy leads to an
improvement in the levels of ALT and AST, which are
serum enzymes indicative of hepatic functions such as
cytolysis and cholestasis

| Liver function | | After 16 weeks treatment | |
|---|---|---|---|
| enzyme | Normal | values | Control values |
| ALT | 0-41 U/L | 59.66 U/L | 84.32 U/L |
| AST | 0-37 U/L | 36.2 U/L | 44.84 U/L |

### Example XII

### Light Microscopic Analysis of Fat Infiltration in the Liver

Formalin-fixed liver samples were stained with hematoxylin-eosin and Oil Red O. Coded histologic slides of animal livers stained with Oil Red O were examined and scored using MATLAB, blinded for the treatment. The scores were as follows: no visible fat: score 0; _upto 10% of liver surface infiltrated by fat: score 1; 10% to 30% fat: score 2; 30% to 50% fat: score 3; and _{—}50% (Note: symbol seems to be missing in front of 50%) and above fat: score 4. Fatty infiltration was classified as microvesicular, macrovesicular, or mixed. Additional findings, such as cellular infiltration and fibrosis, also were recorded.

Fatty Liver Scores. The microscopically determined scores of fatty infiltration decreased markedly in hamsters with the highest dose of FAE microcapsule treatment (Treatment D3-Microencapsulated LF11976 ˜12.98 log cfu/mL). (Table 5). In this group of hamsters the score decreased from 2.9±0.7 to 1.4±0.5, respectively.

FIGS. 14 and 15 show photomicrographs of liver histology from hamsters after 20 weeks on a hypercholesterolemic, hyperlipidemic diet with and without microcapsule treatment. The fat was microvesicular in the hamsters. There was no evidence of inflammation or fibrosis.

TABLE 5
Histological Fatty Liver Scores of Hamsters Fed a
hypercholesterolemic hyperlipidemic diet for 20 weeks

| | Hamsters, 20 Weeks on hypercholesterolemic, | | | |
|---|---|---|---|---|
| | hyperlipidemic diet | | | |
| | Treatment | | | |
| Score | D1 | D2 | D3 | Control |
| 0 | 0 | 0 | 0 | 0 |
| 1 | 0 | 0 | 1 | 0 |
| 2 | 0 | 0 | 3 | 4 |
| 3 | 5 | 3 | 1 | 4 |
| 4 | 0 | 2 | 0 | 2 |
| Mean Score ± S.D. | 2.8 ± 0.4 | 3.2 ± 0.8 | 1.4 ± 0.5 | 2.9 ± 0.7 |

### Example XIII

### Efficacy of the Formulation as an Anti-Oxidant Agent

This formulation is useful for increasing body levels of ferulic acid which is a known antioxidant that neutralizes free radicals (hydrogen peroxide, superoxide, hydroxyl radical and nitrogen dioxide free radicals) which could cause oxidative damage of cell membranes, DNA and accelerated cell aging and is an important factor in diseases such as atherosclerosis and aging. The formulation is useful to prevent cellular damage in clinical situations such as damage caused to body cells by ultraviolet light and others. Exposure to ultraviolet light actually increases the antioxidant potency of ferulic acid and in various anti-aging agents ferulic acid is being used. Similarly this agent is useful as an anti-oxidant, anti-aging supplement in pharmaceutical formulations. Free chemical ferulic acid has been shown to have anti-oxidant effects (Ogiwara, Anticancer Res. 2002; Trombino, J Agric Food Chem. 2004; Graf, Free Radic Biol Med. 1992; Psotova, Biomed Pap Med Fac Univ Palacky Olomouc Czech Repub. 2003).

### Example XIV

### Efficacy of the Formulation as an Anti-Carcinogenic Agent

Various studies are available that show that ferulic acid may have direct anti-tumor activity against different types of cancer. Ferulic acid has pro-apoptotic effects in cancer cells, thereby leading to their destruction. Ferulic acid may be effective at preventing cancer induced by exposure to the carcinogenic compounds benzopyrene and 4-nitroquinoline 1-oxide. Some of the anti-cancer effects appear to be due to the ability of ferulic acid to prevent the conversion of the nitrites used in foods into cancer-causing chemicals. Cancer in a variety of different tissues has been shown to be suppressed by ferulic acid supplementation. These include: cancers of the digestive tract: tongue, esophageal, stomach, intestinal and colorectal cancers; prostate cancer; lung cancer; liver cancer; breast cancer. This formulation therefore is useful in the treatment of cancers as anti-tumoral agent.

### Example XV

### Efficacy of the Formulation as an Lowering Blood Pressure Agent

Researchers working for the Kao Corp. in Japan have discovered that ferulic acid is a potent antihypertensive. In addition, when FA is combined with a diglyceride fat, a stronger hypotensive effect can be achieved. According to the patent, the 15 per cent diglyceride fat and FA compositions can be included in products such as oils, margarine, biscuits and beverages. A daily dose of up to 10 g FA combined with up to 40 g diglyceride may significantly lower blood pressure. (U.S. Pat. No. 6,310,100). Therefore, the present formulation is useful in lowering blood pressure in probiotic, pre-biotic and pharmaceutical formulations.

### Example XVI

### Neuroprotective Agent

In diseases such as Alzheimer's, cognitive decline, macular degeneration. By virtue of its antioxidant properties, ferulic acid greatly reduces free radical damage to the external and internal membranes of nerve cells without causing nerve cell death. Chronic neuroinflammation and oxidative stress contribute to the neurodegeneration associated with Alzheimer's disease and represent targets for therapy. Ferulic acid is a natural compound that expresses antioxidant and anti-inflammatory activities. The free chemical ferulic acid also appears to encourage the proliferation of at least some types of nerve cells, such as retinal cells (Kanski, J Nutr Biochem. 2002; Li, Zhonghua Yan Ke Za Zhi. 2003; Sohn, Arch Pharm Res. 2003). Exploiting these properties, the present formulation can be used for the treatments of Alzheimer's and other neurodegenerative diseases, and in certain clinical situations such as retina and macular degenerations. Also against infantile pathologic conditions such as autism, Attention Deficit Disorder (ADD) and Attention Deficit/Hyperactive Disorder (ADHD).

### Example XVII

### Potential of the Formulation to Prevent Bone Degeneration

Hormone replacement therapy often results in degenerative osteoporosis. Increased levels of ferulic acid in the body can prevent diseases. There is considerable evidence that ferulic acid can be used as supplements to prevent degenerative osteoporosis. Studies of bone metabolism suggest that the free chemical ferulic acid prevents bone loss by a mechanism different from that of estrogens (Sassa, In Vivo. 2003). In an era when hormone replacement therapy is under fire from anti-technology crusaders, this formulation maybe a welcome addition to the osteoporosis treatment arsenal.

### Example XVIII

### Preventing Menopausal Hot Flashes

Agent for prevention of menopausal hot flashes. Free chemical ferulic acid has been shown effective in treating hot flashes in menopausal women (Philp, Altern Med Rev. 2003). This formulation can be used in formulations to treat menopausal hot flashes.

### Example XIX

### Potential of the Formulation as an Agent for Prevention of Diseases by Enhancing Cellular Immunity

Tissue culture experiments have shown that free chemical ferulic acid stimulates the production of human white blood cells and increases the secretion of IFN-gamma (gamma-interferon), an immune-system stimulatory protein. This suggests a possible value of ferulic acid as an immune stimulant, and provides some support for traditional usages of ferulic-acid-containing plants as treatments for cancer and infectious diseases (Chiang, Planta Med. 2003). Therefore, this formulation can be used to enhance cellular immunity.

### Example XX

### Potential of the Formulation as an Agent to Enhance Athletic Performance

Ferulic acid (or its metabolic precursor, gamma oryzanol) has been widely used at a dosage of 250 mg twice per day to enhance athletic performance, both in humans and in race horses (Fry, Int J Sport Nutr. 1997). Thus there is a potential that this formulation can be used as an athletic performance enhancer.

### Example XXI

### Potential of the Formulation in Renal Failure Prevention

The free chemical ferulic acid has a known effect in protecting kidney of diabetic conditions (diabetic nephropathy), improves renal histology, slows or halts progress of renal failure and shows beneficial effect on acute tubular necrosis and fibrosis. It is also known to prevent the formation of renal stones (Zhao, Zhongguo Zhong Xi Yi Jie He Za Zhi. 2004; Liao, Yao Xue Xue Bao. 2003). Thus the present formulation can be used in the prevention of renal failure.

### Example XXII

### Potential of the Formulation as an Effective Agent for Potential Treatment of Ischemic Stroke

The free chemical ferulic acid is shown to improve brain microcirculation through inhibiting thrombus formation and platelet aggregation as well as blood viscosity (Kayahara, Anticancer Res. 1999; Chen, Chin Med J (Engl). 1992). It has been shown to perform as well as drug controls, such as papaverine, dextran and aspirin-persantin. This formulation could thus be potentially used in the treatment of ischemic stroke and blood stasis. The present invention has unique mechanisms [FIG. 16] that are useful in NAFLD and other diseases prevention and therapy. Without wishing to be bound by theory, a reason for the reduction in serum cholesterol concentrations in hamsters fed the high fat high cholesterol diet is that the formulation may inhibit cholesterol absorption, possibly through disruption of the formation of micelles. Another explanation of why the formulation produced significantly lower serum cholesterol in treatment groups as compared to controls is the that the diet is metabolized by the microencapsulated LF11976 supplemented feruloyl esterase enzymes in the GI tract of hamsters into free ferulate and free sterols. The free ferulate is then absorbed and acts as an antioxidant within the plasma, and the free sterol inhibits the cholesterol absorption within the GI tract, thereby lowering blood cholesterol levels. Another reason is the increased fecal excretion of cholesterol and its metabolites.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. An oral formulation to lower serum or hepatic lipid and triglyceride concentrations, hepatic inflammation and/or insulin resistance in a subject in need thereof, which comprises feruloyl esterase producing microorganisms alone or in association with a pharmaceutically acceptable carrier.

2. (canceled)

3. (canceled)

4. (canceled)

5. The oral formulation of claim 1, wherein the microorganisms are encapsulated in polymeric microcapsules wherein said microcapsules are semipermeable.

6. The oral formulation according to claim 1, wherein said feruloyl esterase producing microorganisms are live.

7. The oral formulation according to claim 2, wherein said polymeric microcapsules are resistant to gastrointestinal conditions.

8. The oral formulation according to claim 1, wherein said feruloyl esterase producing microorganisms are wild type, genetically modified, or a combination thereof.

9. The oral formulation of claims 1, wherein said feruloyl esterase producing microorganisms reduce serum or hepatic lipid and triglyceride concentrations by metabolizing food in gastro-intestinal (GI) tract of the subject into free ferulate and free sterols, wherein the free ferulate acts as an antioxidant within liver and/or plasma of the subject and wherein the ferulate inhibits lipid and cholesterol absorption within the GI tract and/or increases fecal excretion of cholesterol and metabolites thereof and results in lipid and cholesterol reduction due to altered regulation of liver enzymes.

10. The oral formulation of claim 1, wherein said feruloyl esterase producing microorganisms are selected from the group consisting of bacteria, yeast and combinations thereof.

11. The oral formulation according to claim 1, wherein said feruloyl esterase producing microorganisms are wild type feruloyl esterase producing Lactobacillus, Bifidobacteria or Bacillus bacterial cells, or feruloyl esterase producing genetically engineered cells and yeast cells.

12. (canceled)

13. The oral formulation according to claim 11, wherein said feruloyl esterase producing Lactobacillus or Bacillus bacterial cells are selected from the group consisting of Lactobacillus fermentum, Lactobacillus reuteri and Lactobacillus farciminis.

14. The oral formulation according to claims 1, wherein said pharmaceutically acceptable carrier comprises fermented milk.

15. The oral formulation according to claim 14, wherein said fermented milk carrier comprises a basic pH buffer and protects the feruloyl esterase producing microorganisms from gastrointestinal fluids.

16. (canceled)

17. The oral formulation according to claim 14, wherein said fermented milk carrier comprises a food supplement or food.

18. The oral formulation according to claim 17, wherein said food or food supplement comprises yogurt, cheese, milk, powdered milk, cream, butter, ice cream, kefer or a fermented milk formulation.

19. (canceled)

20. The oral formulation according to claim 18, wherein the yogurt comprises 1-10 grams of feruloyl esterase producing microorganisms per 100 grams of yogurt.

21. (canceled)

22. (canceled)

23. The oral formulation according to claim 1, which further comprises dietary fiber rich in polyphenols and hesperetin metabolites, whole grains and bran, phtochemicals, probiotics, psyllium, phytosterols, coffee beans, grapes, apples, artichokes, oranges, pineapple, peanuts, vitamins and/or antibiotics.

24. (canceled)

25. (canceled)

26. (canceled)

27. (canceled)

28. (canceled)

29. (canceled)

30. (canceled)

31. (canceled)

32. The oral formulation according to claim 2, wherein said microcapsules are made of a material chosen from Alginate-Poly-L-lysine-Alginate [APA], Alginate-Chitosan [AC], Alginate-Chitosan-Polyethylene glycol (PEG)-Poly-L-lysine (PLL)-Alginate [ACPPA], Alginate -Poly-L-lysine-PEG-Alginate [APPA], Alginate-Chitosan-PEG [ACP], Alginate-Poly-L-lysine-Pectinate-Poly-L-lysine-Alg inate [APPPA], Genipin cross-linked alginate-chitosan (GCAC).

33. The oral formulation according to claim 2, wherein said microcapsules are made of Alginate-Poly-L-lysine-Alginate [APA].

34. The oral formulation according to claim 1, which is formulated as a tablet, a capsule, a jellified tablet, a caplet and a liquid formulation.

35. (canceled)

36. (canceled)

37. (canceled)

38. (canceled)

39. (canceled)

40. (canceled)

41. (canceled)

42. (canceled)

43. (canceled)

44. A method for preventing or treating a disease or disorder associated with high serum or hepatic lipid and triglyceride concentrations, hepatic inflammation and/or insulin resistance in a subject, which comprises orally administering to the subject a sufficient amount of the oral formulation of claim 1.

45. The method according to claim 44, wherein said disease or disorder comprises non-alcoholic fatty liver disease (NAFLD), alcoholic fatty liver disease (AFLD), liver cirrhosis, liver steatosis, liver fibrosis, hyperlipidemia, hypercholesterolemia, hyperlipoproteinemia, hypertriglyceridemia, atherosclerosis, abnormally high serum ALT, AST and GGT levels, obesity, type II diabetes, Epstein-Barr virus, type I diabetes, hepatitis, autoimmune hepatitis, hepatic granulomatus disease, tuberculosis, cholangitis, hepatocellular cancer, cholangiocarcinoma, non-alcoholic steatohepatitis (NASH), haemochromatosis, Wilson's disease, Gilbert's syndrome, Crigler-Najjar syndrome, Dubin-Johnsons syndrome or Reye's syndrome.

46. (canceled)

47. The method according to claim 44, wherein said disease or disorder comprises sequelae of acute hyperglycemia and/or increased fatty acid flux in subject and wherein preventing said diseases and disorders prevents metabolite-induced reactive oxygen-species mediated injury.

48. (canceled)

49. A method of enhancing anti-oxidant agent activity comprising orally administering to a subject a sufficient amount of the oral formulation of claim 1.

50. (canceled)

51. A method for treatment and/or prevention of a disease or disorder selected from the group consisting of cancer of the digestive tract, colorectal cancer, prostate cancer, lung cancer, liver cancer, breast cancer, Alzheimer's disease, cognitive decline, macular degeneration, high blood pressure, tumours, osteoporosis, menopausal hot flashes, renal failure and ischemic stroke which comprises orally administering a sufficient amount of the oral formulation of claim 1.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A method of improving brain microcirculation through inhibiting thrombus formation and platelet aggregation as well as reducing blood viscosity comprising orally administering a sufficient amount of the oral formulation of claim 1.

64. (canceled)

65. The oral formulation of claim 1, wherein said feruloyl esterase producing microorganisms are present in a range from 10⁶ to 10¹³ colony forming units per millimeter (CFU/mL).

66. The oral formulation of claim 2, wherein said polymeric microcapsules comprise crude or purified bacterial fermentation broth,

67. (canceled)

68. A method for preventing or treating a disease or disorder associated with high serum or hepatic lipid and triglyceride concentrations, hepatic inflammation and/or insulin resistance in a subject, which comprises orally administering to the subject a sufficient amount of an oral formulation comprising feruloyl esterase producing microorganisms alone or in association with a pharmaceutically acceptable carrier.

69. A method of reducing cholesterol in a subject comprising orally administering a sufficient amount of the oral formulation of claim 1.

70. A method of lowering blood glucose levels in a subject comprising orally administering a sufficient amount of the oral formulation of claim 1.

71. A method of reducing triglycerides in a subject comprising orally administering a sufficient amount of the oral formulation of claim 1.
